(19) United States
(12) Patent Application Publication
(10) Pub. No.: US 2026/0207554 A1
(43) Pub. Date: Jul. 23, 2026

(54) REGIMENS AND COMPOSITIONS USEFUL FOR ALLEVIATING PAIN

(71) Applicant: PARK THERAPEUTICS, INC., Monmouth junction, NJ (US)

(72) Inventors: James Hackworth, Morristown, NJ (US); Marc Lesnick, Knoxville, TN (US); Antonio Pardo, New York, NY (US); Joseph Grieco, Kinnelon, NJ (US)

(73) Assignee: Adneuris Therapeutics, Inc., Monmouth Junction, NJ (US)

(21) Appl. No.: 19/137,743

(22) PCT Filed: Dec. 12, 2023
(86) PCT No.: PCT/US2023/083697

§ 371 Date: Jun. 11, 2025

Related U.S. Application Data
(60) Provisional application No. 63/387,086, filed on Dec. 12, 2022.
(60) Provisional application No. 63/485,139, filed on Feb. 15, 2023.
(60) Provisional application No. 63/578,894, filed on Aug. 25, 2023.

(51) Int. Cl. A61K 31/407

(52) U.S. Cl. CPC A61K 31/407

(57) ABSTRACT

Provided herein are methods for treating patients, including those in need of analgesic treatment, and reducing abuse potential and reducing, eliminating and/or preventing hot flushing, hyperhidrosis, and/or pruritis side effects of opioids and opioid-like compounds (e.g., analgesics in Class II, III and Class IV) in a treated patient. The method comprises dosing a patient once a day with an immediate release composition comprising cebranopadol in crystal form A and/or in free base form.

### BACKGROUND OF THE INVENTION

Opioid agonists provide analgesic effects by acting on opioid receptors in the central and peripheral nervous systems that block the sensation of pain from signaling to the brain. Opioid agonists are available in different dosage forms. Opioid agonists may be characterized as full agonist opioids or partial agonist opioids. However, undesirable common side effects of opioid administration include sedation, dizziness, nausea, vomiting, constipation, physical dependence, tolerance, and respiratory depression.

Cebranopadol (trans-6′-fluoro-4′,9′-dihydro-N,N-dimethyl-4-phenyl-spiro[cyclohexane-1,1′-(3′H)-pyrano[3,4-b]indol]-4-amine) is an analgesic nociceptin/orphanin FQ peptide (NOP) and opioid receptor agonist (WO 2004/043967, WO 2008/040481, WO 2012/016703, WO 2012/016699, WO 2012/016695, WO 2012/016698, WO 2012/016697, WO 2013/007361). Cebranopadol exhibits highly potent and efficacious antinociceptive and antihypersensitive effects in several rat models of acute and chronic pain with ED50 values of 0.5-5.6 μg/kg after intravenous and 25.1 μg/kg after oral administration. In comparison with selective mu (u)-opioid receptor (MOP) agonists, cebranopadol was more potent in models of chronic neuropathic than acute nociceptive pain.

Cebranopadol displays broad activity in various pain states and is highly potent and efficacious in animal models of acute nociceptive, inflammatory, cancer, and, especially, chronic neuropathic pain. In contrast to opioids such as morphine, cebranopadol displays higher analgesic potency in chronic pain, especially of neuropathic origin, than in acute nociceptive pain. In addition, even after doses higher than those required for inducing analgesia, cebranopadol affects neither motor coordination nor respiratory function and thus displays a better tolerability profile than opioids. As a result, there is a broader therapeutic window for cebranopadol than for morphine (K. Linz et al., J. Pharmacol. Exp. Ther. 2014 535-548).

There remains a need for improved therapies that do not carry the well-established risks associated with traditional opioids used for alleviating pain in patients.

### BRIEF DESCRIPTION OF THE FIGURES

FIG. 1 shows results of a single-dose, randomized, double-blind, placebo- and active-controlled crossover trial of oral human-abuse potential study, graphed as a difference in Drug Liking (VAS) scores in modified completer population.

FIG. 2 provides shows results of a single-dose, randomized, double-blind, placebo- and active-controlled crossover trial of oral human-abuse potential study, graphed as a difference in Drug Liking (VAS) scores in completer population.

FIG. 3 shows drug liking visual analog scale (VAS).

FIG. 4 shows cebranopadol-induced respiratory depression in healthy volunteers.

FIG. 5 shows mean drug liking time course profile.

FIG. 6A shows cebranopadol effect on rat heroin self-administration.

FIG. 6B shows cebranopadol effect on rat heroin self-administration.

FIG. 7A shows drug Liking (at this moment) VAS-Summary Parameters by Treatment During the Treatment Phase. Modified Completer Population. T_{Emax} and T_{Emin} are reported in hours.

FIG. 7B shows drug Liking (at this moment) VAS-Summary Parameters by Treatment During the Treatment Phase. Modified Completer Population. T_{Emax} and T_{Emin} are reported in hours.

FIG. 8A shows drug Liking (at this moment) VAS-Summary Parameters by Treatment During the Treatment Phase. Completer Population. T_{Emax} and T_{Emin} are reported in hours.

FIG. 8B shows drug Liking (at this moment) VAS-Summary Parameters by Treatment During the Treatment Phase. Completer Population. T_{Emax} and T_{Emin} are reported in hours.

FIG. 9 shows drug Liking (at this moment) VAS-Analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 10D shows drug Liking (at this moment) VAS-Analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP≥11 vs ha: μT-μP<11, where IT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 11 shows drug Liking (at this moment) VAS-Analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP≥11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 12 shows drug liking (at this moment) VAS-analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 13 shows drug liking (at this moment) VAS-Analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 14 shows drug liking (at this moment) VAS-Analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP≥11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 15 shows drug liking (at this moment) VAS-analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 16 shows drug liking (at this moment) VAS-analysis results. Modified Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 17 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 18 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 19 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 20 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: C2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP≥11 vs ha: μT-μP<11, where μT is the mean for cebranopadol and μP is mean for placebo; For all other analyses, the hypotheses are the same as above, but with margins of 0 and the cebranopadol versus placebo comparison hypothesis is two-sided. Sign tests hypotheses test medians rather than means.

FIG. 21 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 22 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: AT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 23 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: μC2-μT≤0 vs ha: μC2-μT >0, where μC2 is the mean for tramadol IR and μT is mean for cebranopadol; Testing h0: μT-μP≥11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 24 shows drug liking (at this moment) VAS-analysis results. Completers Population. P-values≤0.05 are bolded. For the primary analysis of Drug Liking Emax, the hypothesis tested are as follows: Testing h0: μC1-μP≤15 vs ha: μC1-μP>15, where μC1 is the mean for oxycodone IR and μP is mean for placebo; Testing h0: μC2-μP≤15 vs ha: μC2-μP>15, where μC2 is the mean for tramadol IR and μP is mean for placebo; Testing h0: μC1-μT≤0 vs ha: μC1-μT >0, where μC1 is the mean for oxycodone IR and μT is mean for cebranopadol; Testing h0: C2-μT≤0 vs ha: μC2-μT >0, where μC2 is the mean for tramadol IR and μT is mean for cebranopadol; Testing h0: μT-μP>11 vs ha: μT-μP<11, where μT is the mean for cebranopadol and μP is mean for placebo. For all other analyses, the hypotheses are the same as above, but with margins of 0 and the cebranopadol versus placebo comparison hypothesis is two-sided. Sign tests hypotheses test medians rather than means.

FIG. 25 shows overall summary of treatment emergent adverse events (TEAEs). Safety Population. Abbreviations: AE=adverse event, TEAE=treatment emergent adverse event, SAE=serious adverse event. All AEs were coded using the Medical Dictionary for Regulatory Activities (MedDRA), Version 24.1. Percentages are based on the number of subjects in the Safety Population. For patient counts, if a subject experienced one or more events, the subject is counted only once in the subject count. TEAE is defined as any new AE that occurs after dosing of study medication until End of Study or Early Termination.

FIG. 26 shows summary of treatment emergent adverse events, by system organ class and preferred term. Safety Population. Abbreviations: TEAE=treatment emergent adverse event. A TEAE is defined as any new AE that occurs after dosing of study medication until End of Study or Early Termination. At each level of summarization (System Organ Class or Preferred Term), subjects who have more than one adverse event were only counted once. All adverse events were coded using the Medical Dictionary for Regulatory Activities (MedDRA), Version 24.1. System Organ Classes and Preferred Terms are sorted in order of frequency. Percentages are based on the number of subjects in the Safety Population.

FIG. 27 shows summary of treatment emergent adverse events, by system organ class and preferred term. Safety Population. Abbreviations: TEAE=treatment emergent adverse event. A TEAE is defined as any new AE that occurs after dosing of study medication until End of Study or Early Termination. At each level of summarization (System Organ Class or Preferred Term), subjects who have more than one adverse event were only counted once. All adverse events were coded using the Medical Dictionary for Regulatory Activities (MedDRA), Version 24.1. System Organ Classes and Preferred Terms are sorted in order of frequency. Percentages are based on the number of subjects in the Safety Population.

FIG. 28 shows summary of treatment emergent adverse events, by system organ class and preferred term. Safety Population. Abbreviations: TEAE=treatment emergent adverse event. A TEAE is defined as any new AE that occurs after dosing of study medication until End of Study or Early Termination. At each level of summarization (System Organ Class or Preferred Term), subjects who have more than one adverse event were only counted once. All adverse events were coded using the Medical Dictionary for Regulatory Activities (MedDRA), Version 24.1. System Organ Classes and Preferred Terms are sorted in order of frequency. Percentages are based on the number of subjects in the Safety Population.

FIG. 29 shows summary of treatment emergent adverse events, by system organ class and preferred term. Safety Population. Abbreviations: TEAE=treatment emergent adverse event. A TEAE is defined as any new AE that occurs after dosing of study medication until End of Study or Early Termination. At each level of summarization (System Organ Class or Preferred Term), subjects who have more than one adverse event were only counted once. All adverse events were coded using the Medical Dictionary for Regulatory Activities (MedDRA), Version 24.1. System Organ Classes and Preferred Terms are sorted in order of frequency. Percentages are based on the number of subjects in the Safety Population.

FIG. 30 shows summary of treatment emergent adverse events, by system organ class and preferred term. Safety Population. Abbreviations: TEAE=treatment emergent adverse event. A TEAE is defined as any new AE that occurs after dosing of study medication until End of Study or Early Termination. At each level of summarization (System Organ Class or Preferred Term), subjects who have more than one adverse event were only counted once. All adverse events were coded using the Medical Dictionary for Regulatory Activities (MedDRA), Version 24.1. System Organ Classes and Preferred Terms are sorted in order of frequency. Percentages are based on the number of subjects in the Safety Population.

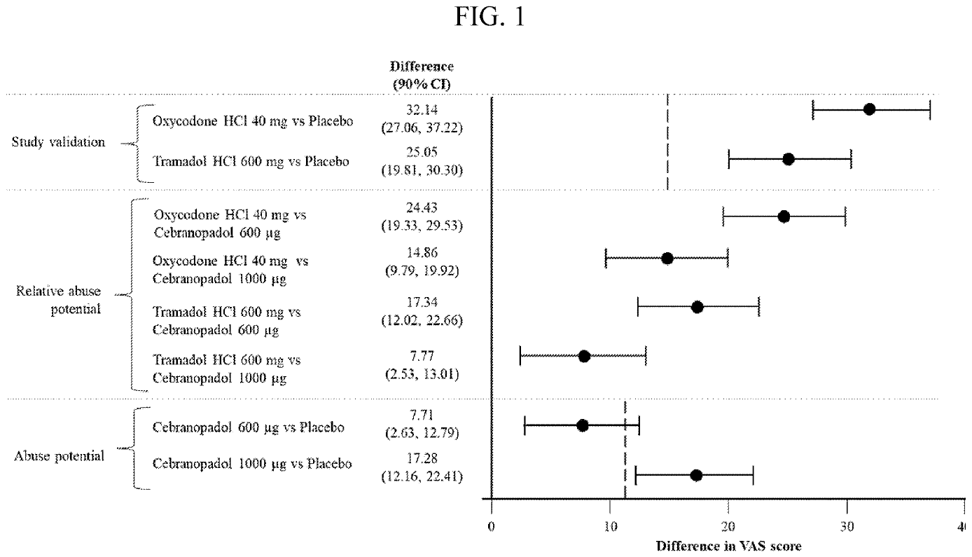

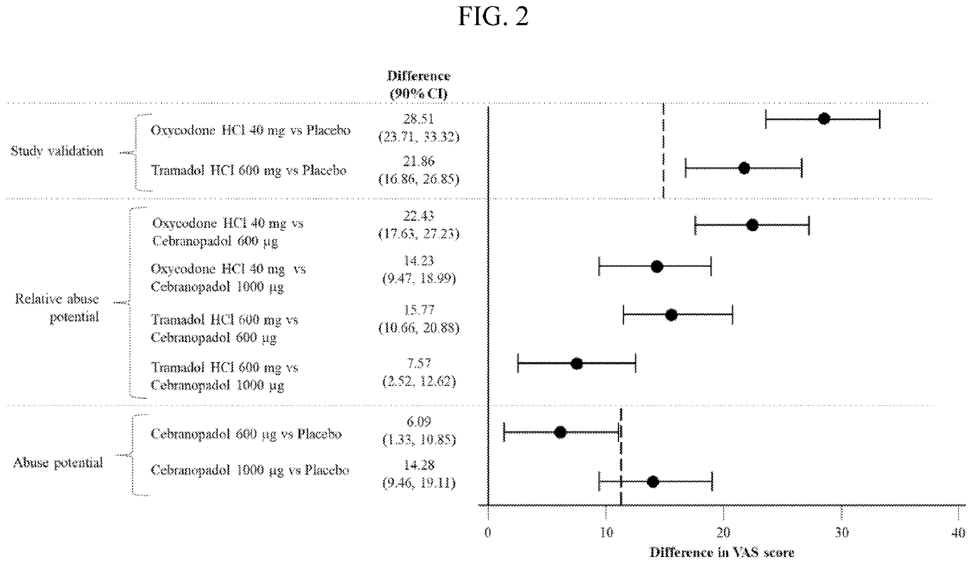

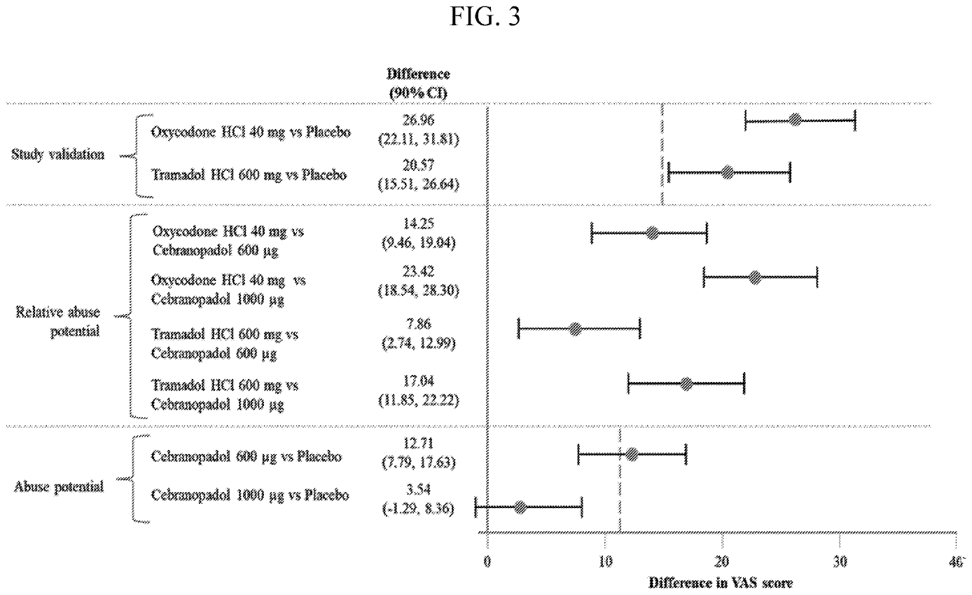

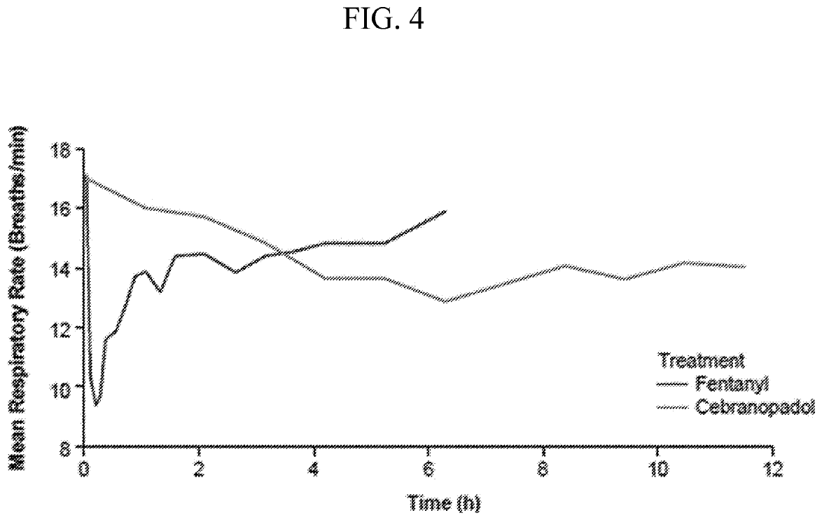

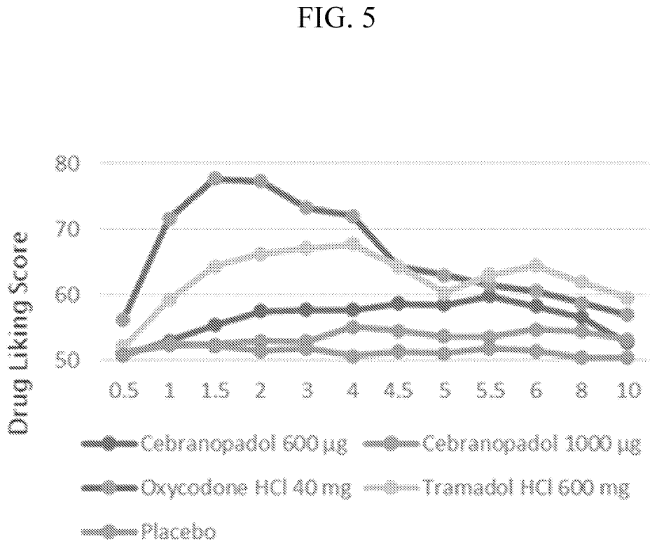

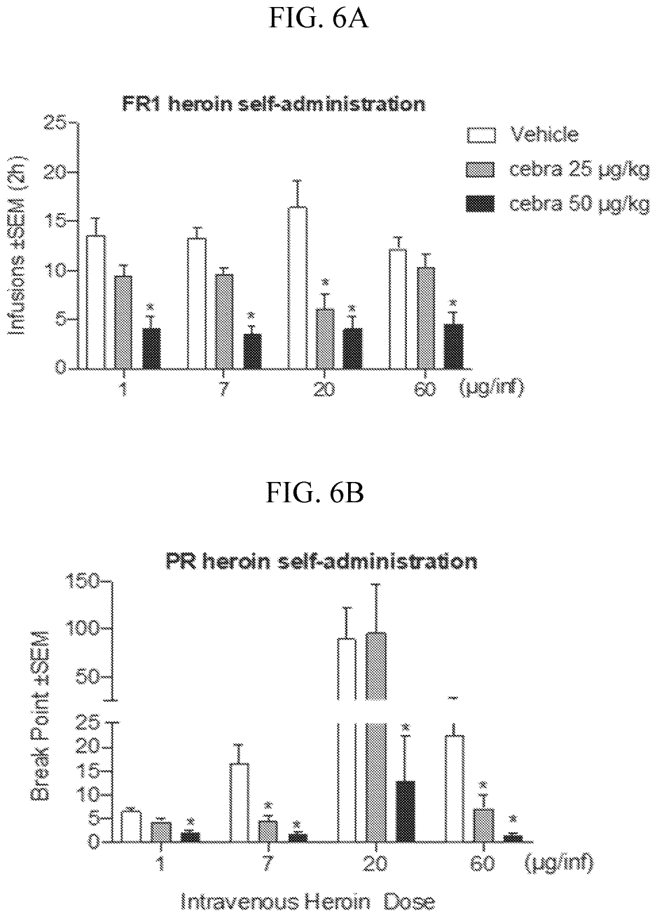

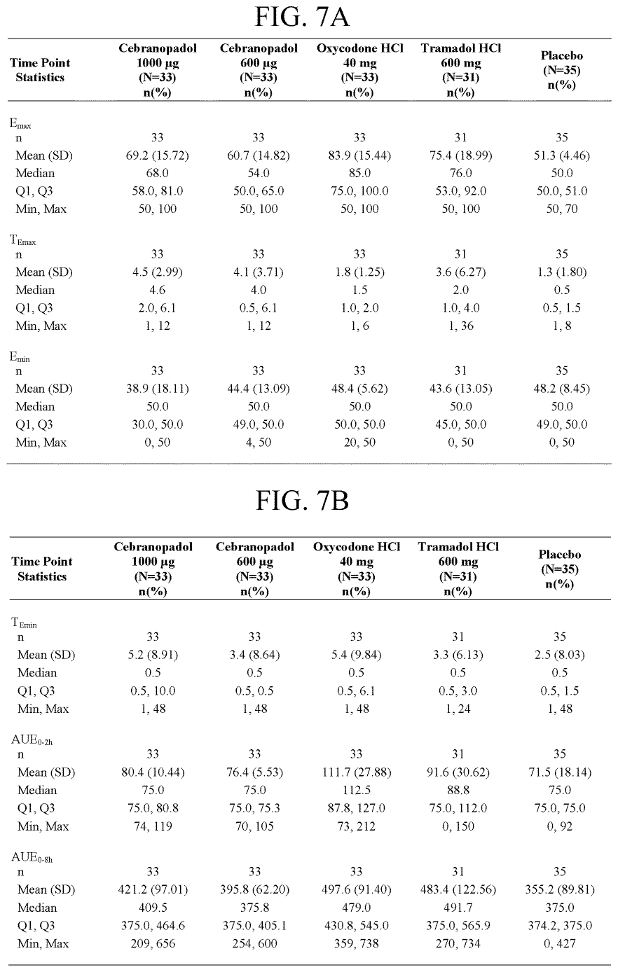

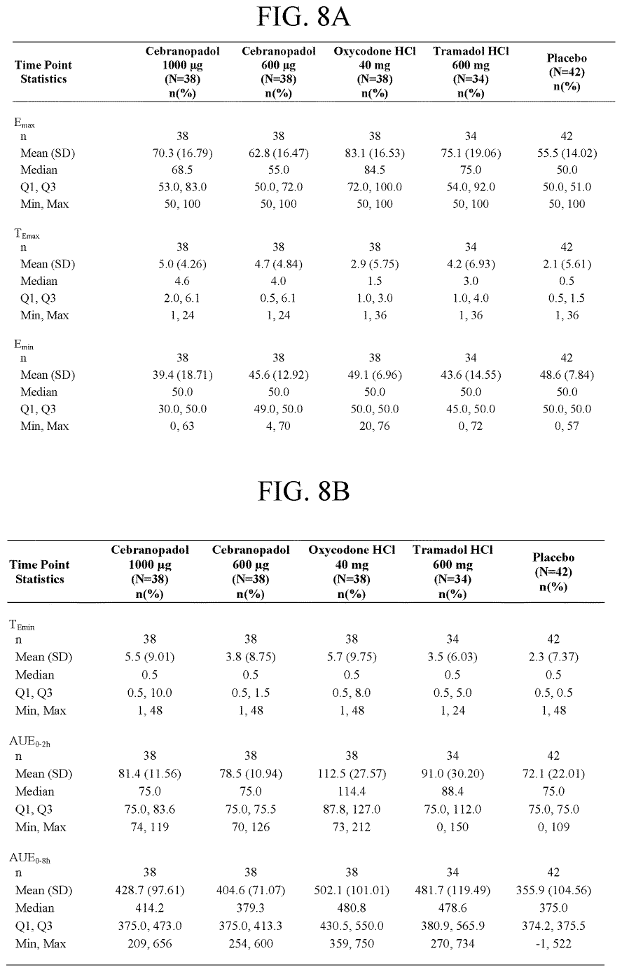

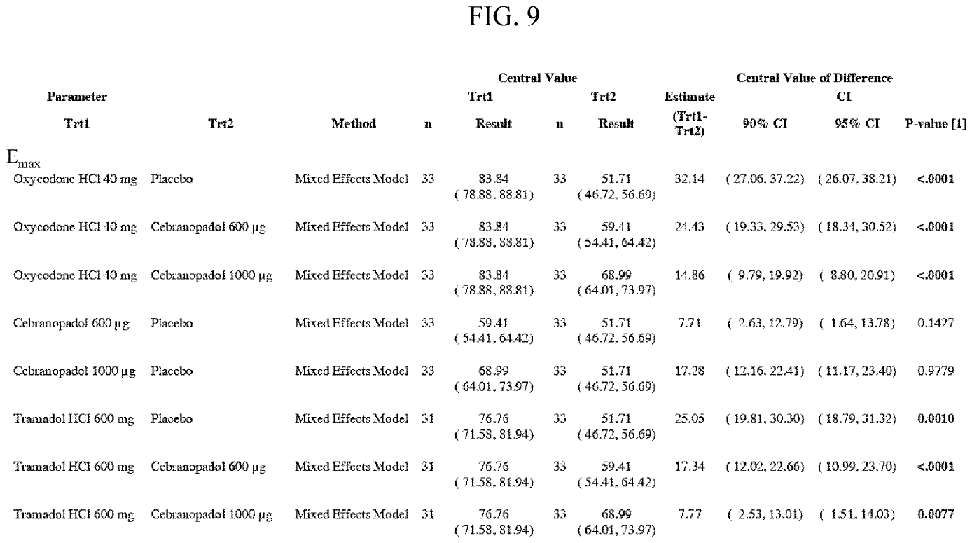

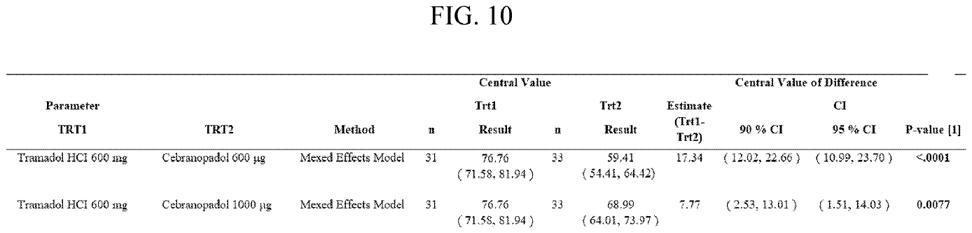

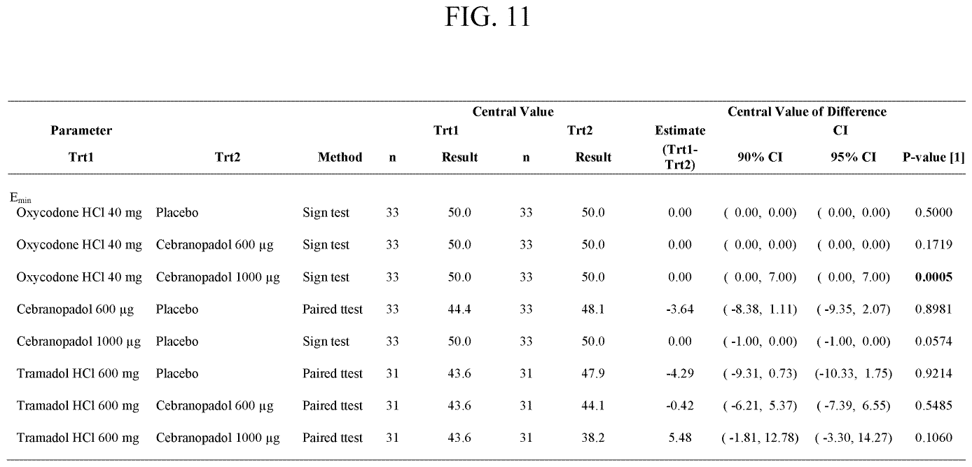

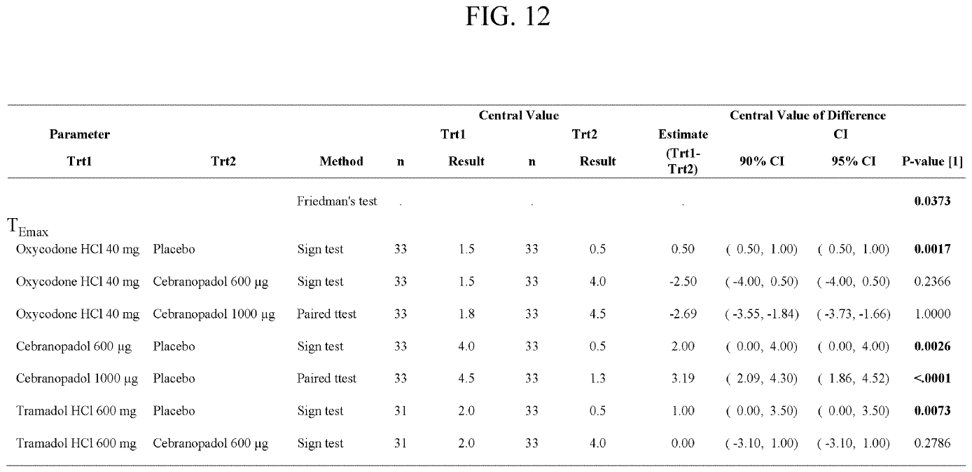

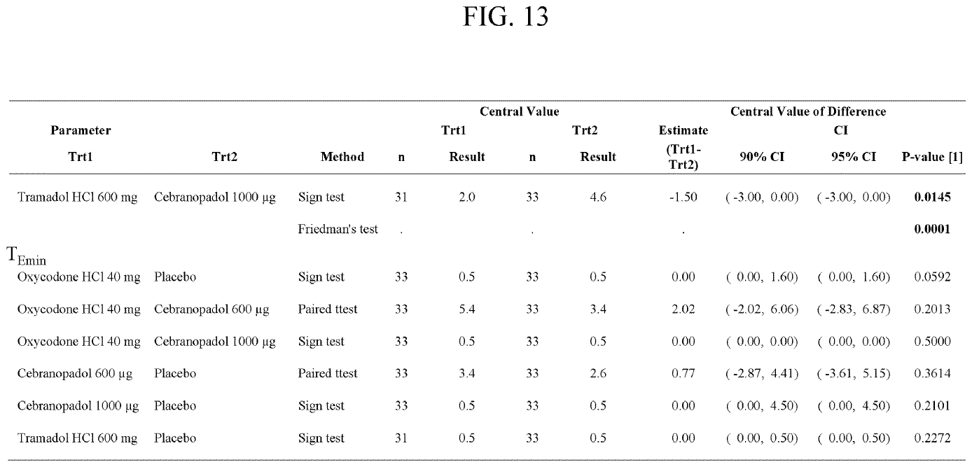

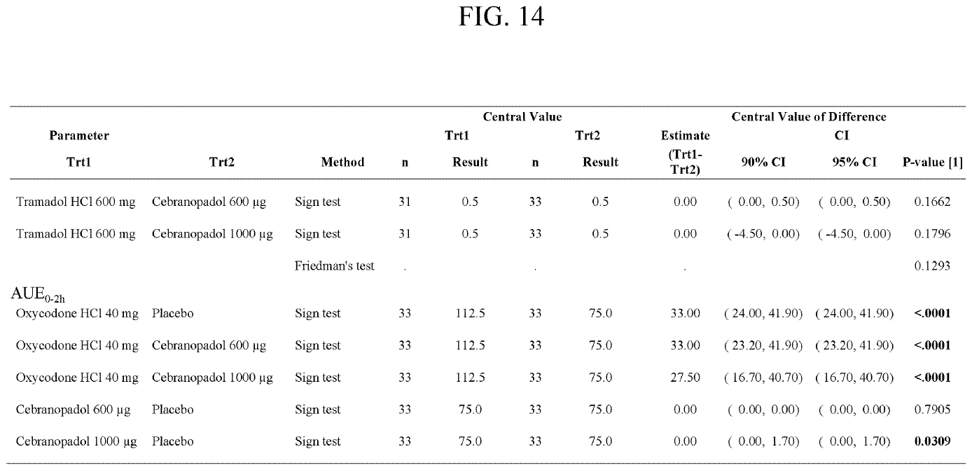

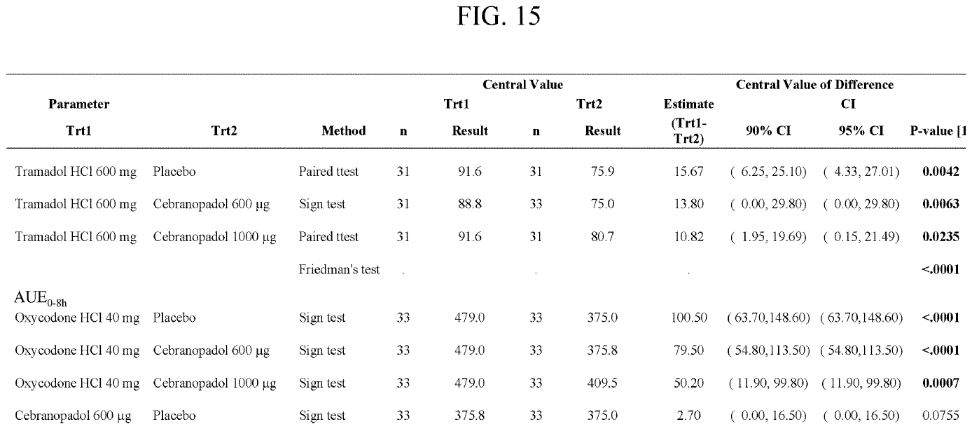

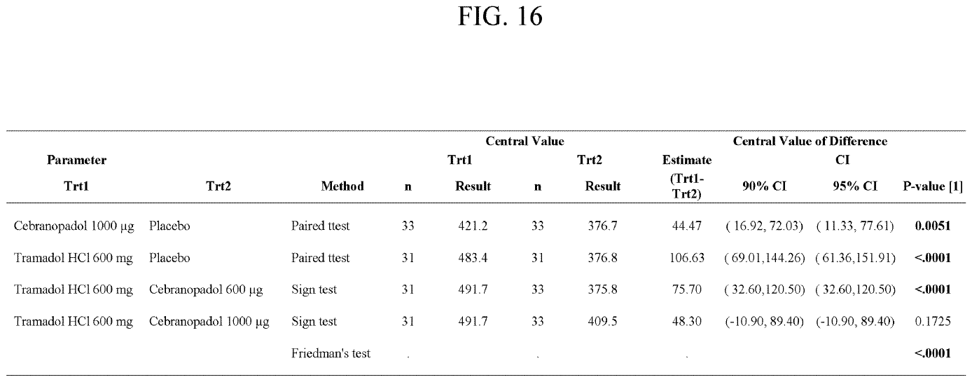

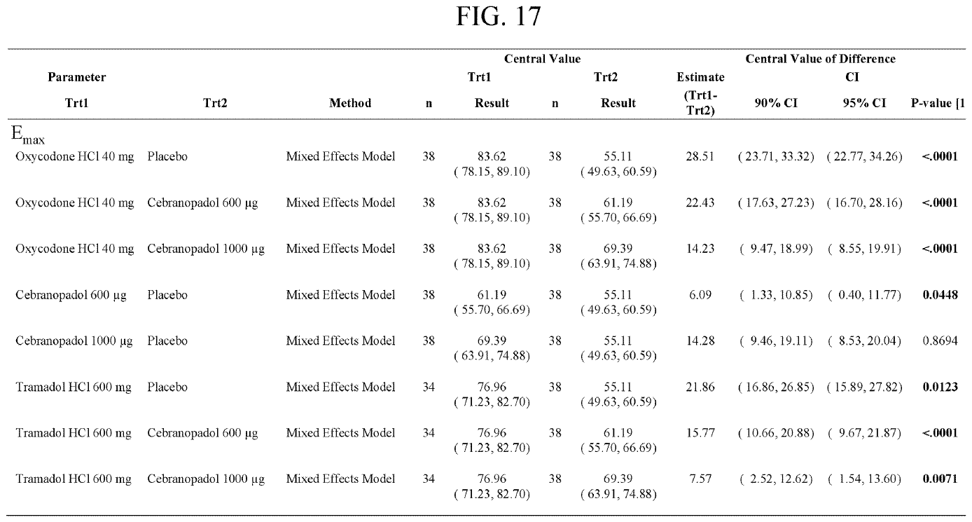

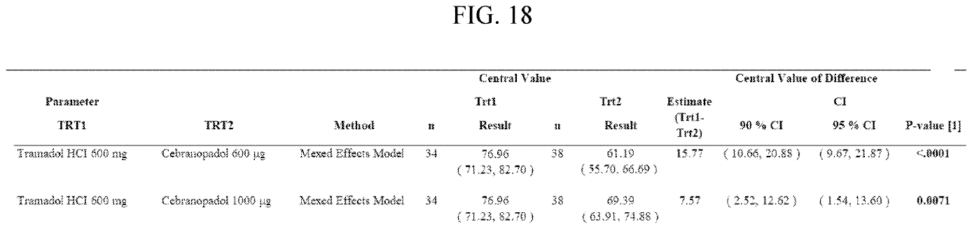

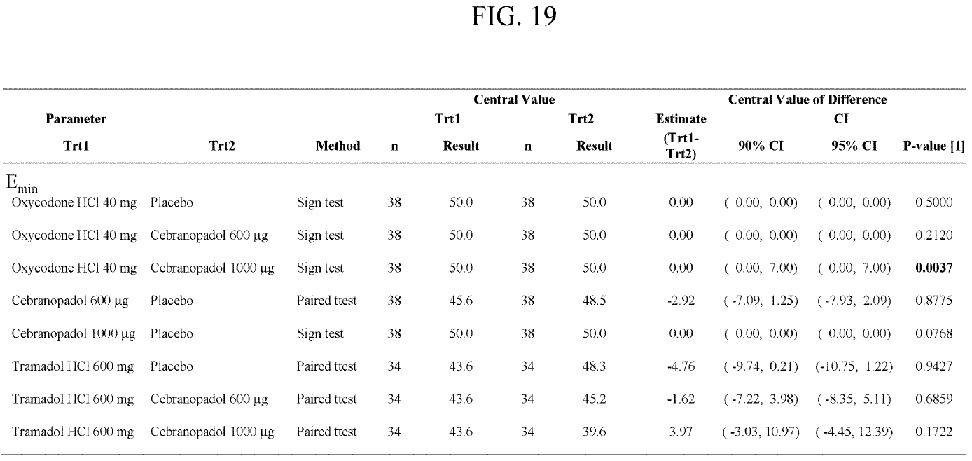

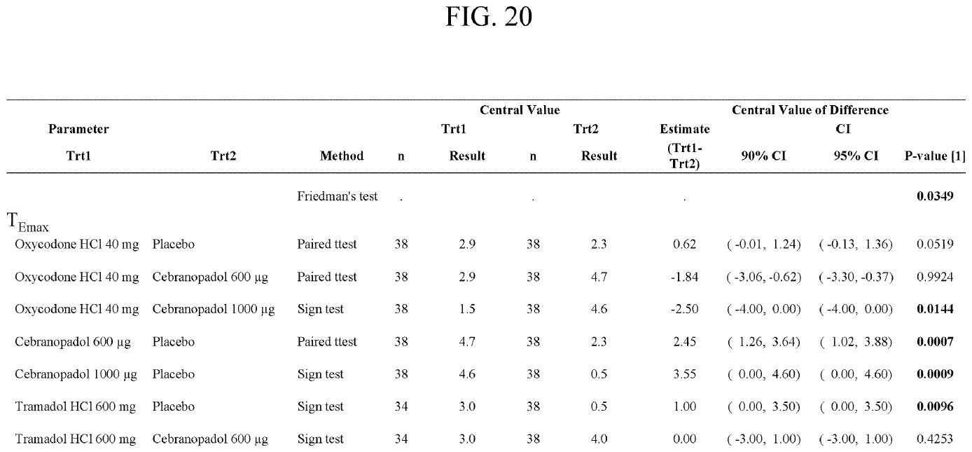

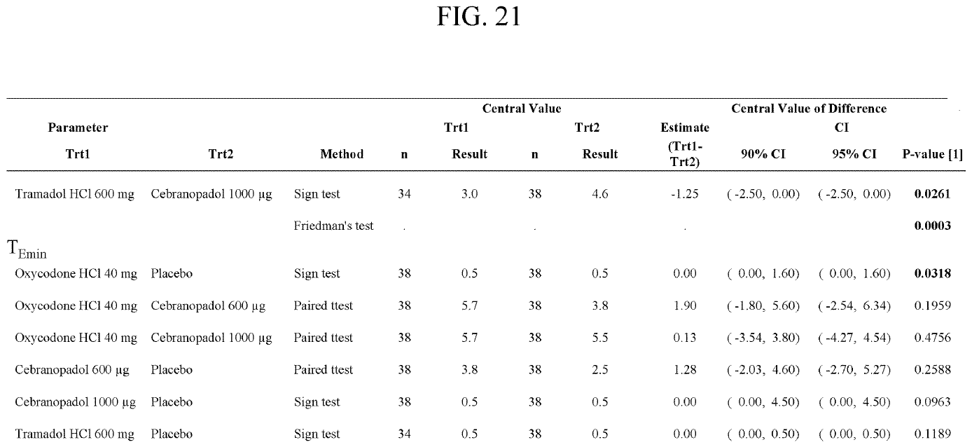

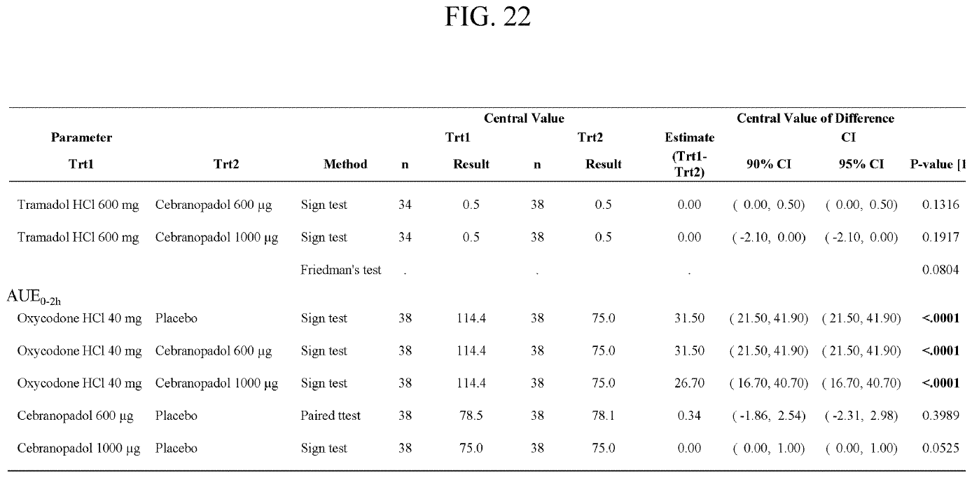

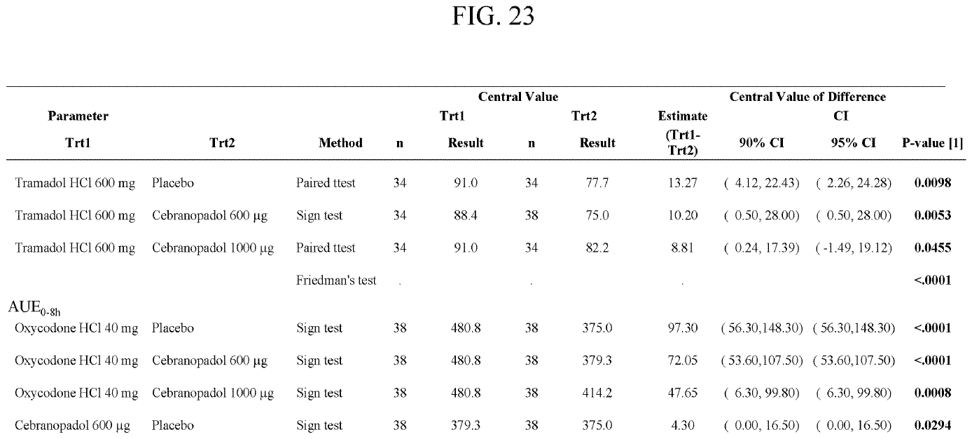

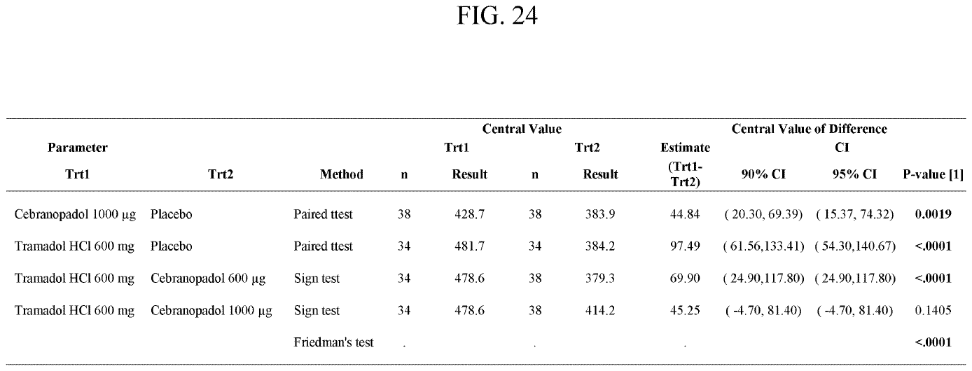

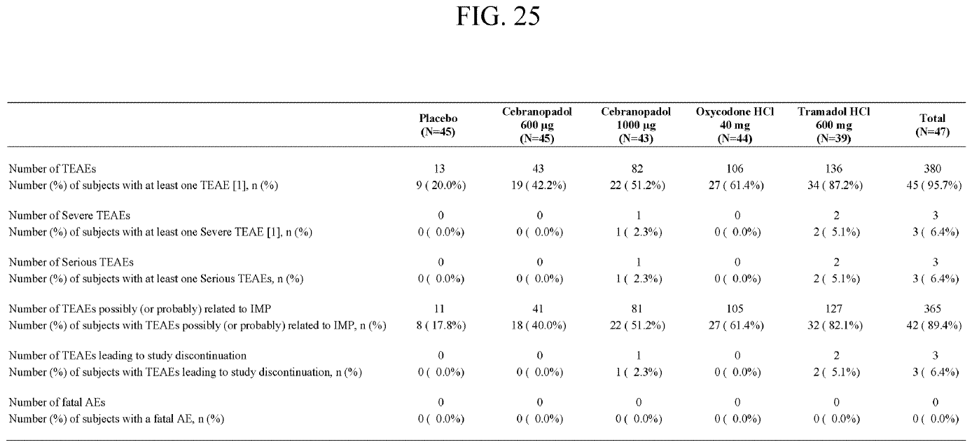

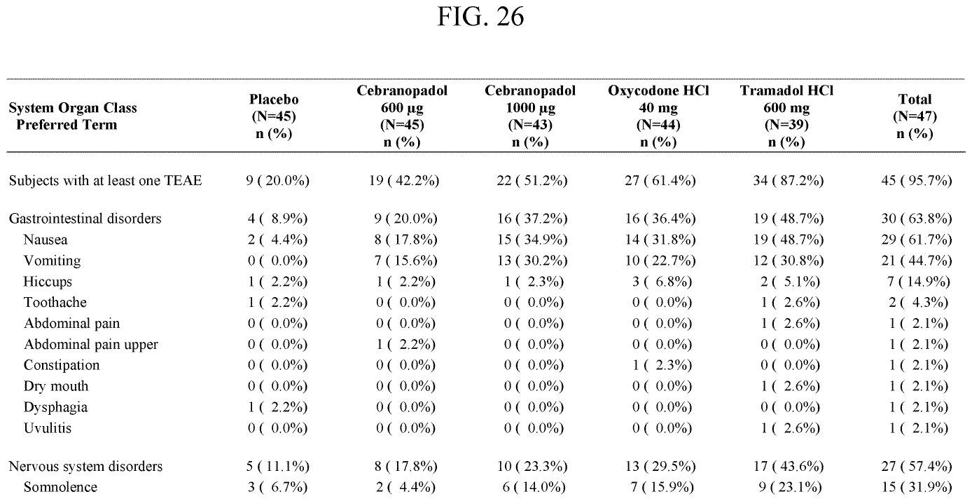

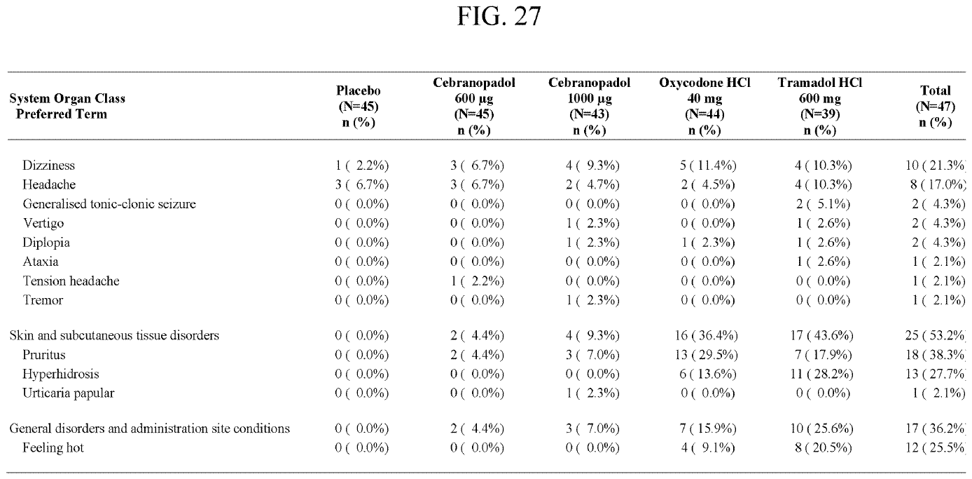

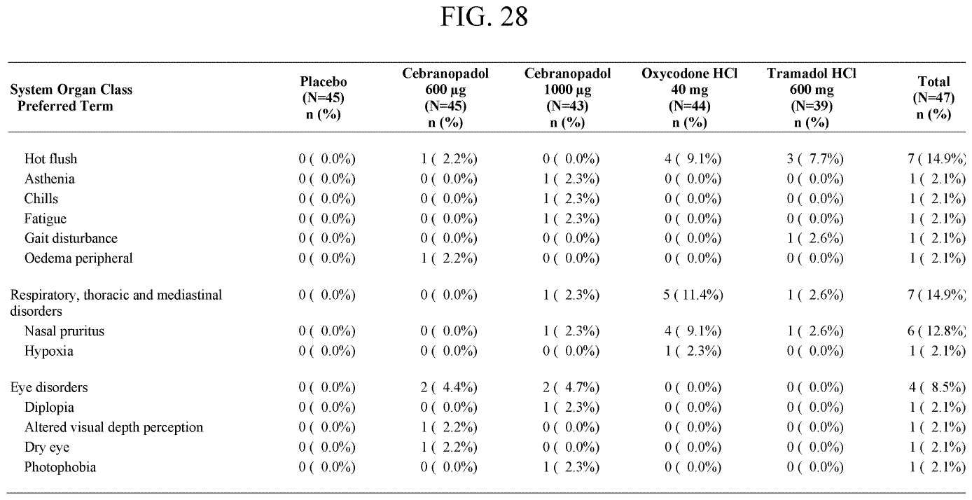

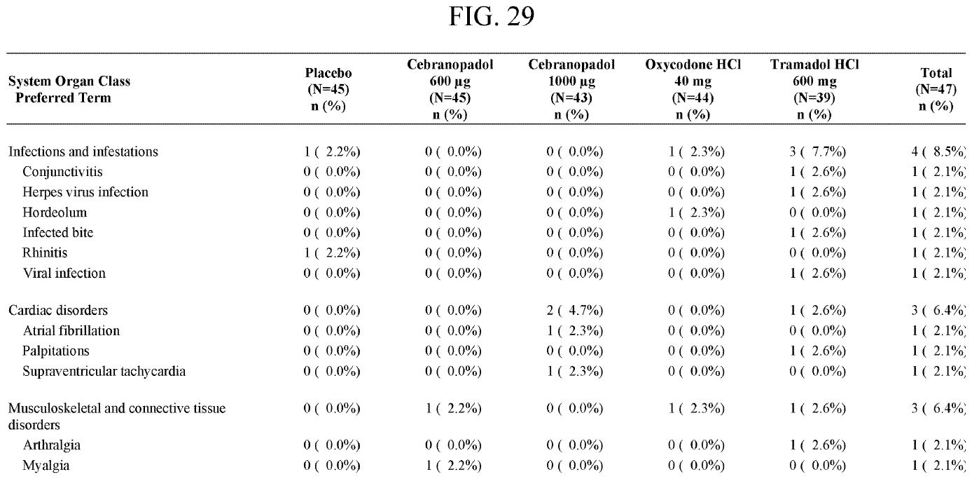

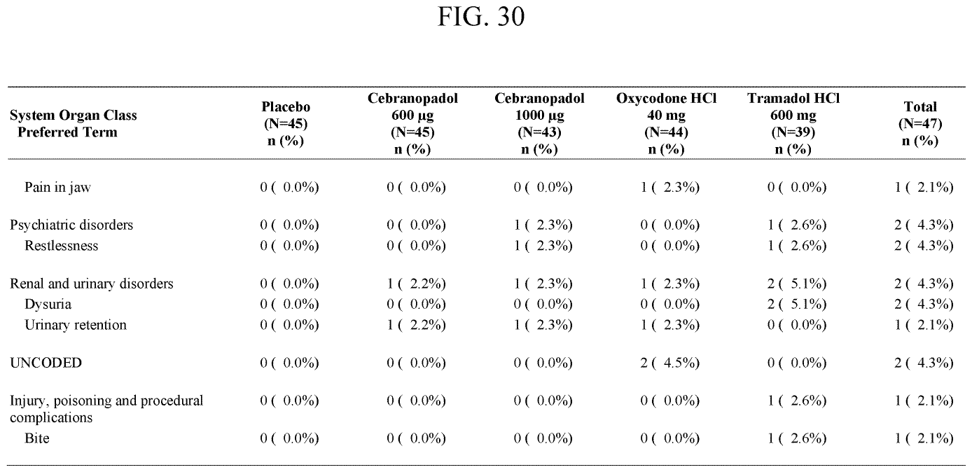

### SUMMARY OF THE INVENTION

In certain embodiments, a composition is provided which is useful in treating a subject, wherein the composition comprises cebranopadol or a pharmaceutically acceptable salt, hydrate, or salt hydrate, which when administered to a subject provides the subject with a therapeutic effect of a pharmaceutically acceptable full mu-agonist and a lower potential for abuse than a pharmaceutically acceptable partial mu-agonist. In certain embodiments, the subject is being treated for pain. In certain embodiments, the pain is chronic; acute; central; peripheral; neuropathic and/or nociceptive pain. In certain embodiments, the pain is visceral pain, skeletal pain, and/or nervous pain. In certain embodiments, the dose is administered daily in an amount of about 10 ug to about 2000 ug cebranopadol. In certain embodiments, cebranopadol is a free base. In certain embodiments, at least 80% of the cebranopadol is in crystal form A. In certain embodiments, the partial mu-agonist is tramadol. In certain embodiments, use of cebranopadol in treating a subject is provided, wherein the composition comprises cebranopadol or a pharmaceutically acceptable salt, hydrate, or salt hydrate, which when administered to a subject provides the subject with a full mu-agonist therapeutic effect and a lower abuse potential than a partial mu-agonist. In certain embodiments, use of cebranopadol in preparing a medicament for treating a subject is provided, wherein the composition comprises cebranopadol or a pharmaceutically acceptable salt, hydrate, or salt hydrate, which when administered to a subject provides the subject with a full mu-agonist therapeutic effect and a lower abuse potential than a partial mu agonist.

In certain embodiments, a composition, use or method is provided for treating a subject, wherein the composition comprises cebranopadol or a pharmaceutically acceptable salt, hydrate, salt hydrate, which when administered to a subject provides the subject with a full mu-agonist therapeutic effect and a lower abuse potential than a partial mu-agonist. In certain embodiments, the subject is being treated for pain. In certain embodiments, the pain is chronic; acute; central; peripheral; neuropathic and/or nociceptive pain. In certain embodiments, the pain is visceral pain, skeletal pain, and/or nervous pain. In certain embodiments, the mu-agonist activity is assessed using a visual analog scale (VAS rating), pupillometry, and/or a Multi-Task Test. In certain embodiments, the dose is administered daily in an amount of about 10 μg to about 2000 μg cebranopadol. In certain embodiments, the cebranopadol is a free base. In certain embodiments, at least 80% of the cebranopadol is in crystal form A. In certain embodiments, the partial mu-agonist is tramadol.

In certain embodiments, a composition, use or method treating pain in a patient having nociceptive pain with reduced risk of abuse is provided, said regimen comprising dosing a patient once daily with an immediate release composition comprising cebranopadol or a pharmaceutically acceptable salt thereof. In certain embodiments, the composition is a film-coated tablet. In certain embodiments, the cebranopadol is in free base form. In certain embodiments, at least 80% of the cebranopadol in the composition is in crystal form A.

In certain embodiments, a composition, use or method is provided which is useful for reducing pruritus, hyperhidrosis, feeling hot and/or hot in a subject receiving analgesic treatment for pain, while providing the analgesic therapeutic effect of an opioid, wherein the composition comprises cebranopadol. In certain embodiments, the cebranopadol is in free base form. In certain embodiments, at least 80% of the cebranopadol in the composition is in crystal form A.

In certain embodiments, a composition or regimen provided herein comprises cebranopadol as the sole active pharmaceutical ingredient or the sole analgesic in the composition.

In certain embodiments, a composition, use or method is provided for reducing the abuse potential and sides effects of Class II, III and Class IV-opioids and opioid-like analgesics in a patient in need of analgesic treatment, said method comprising dosing a patient once a day with an immediate release composition comprising cebranopadol or a pharmaceutically acceptable salt or hydrate thereof. In certain embodiments, the composition is an immediate release composition administered once daily and provides an analgesic effect over a period of at least about 8 to 24 hour hours post-administration. In certain embodiments, the pain is visceral pain, skeletal pain, and/or nervous pain. In certain embodiments, the mu-agonist activity is assessed using a visual analog scale (VAS rating), pupillometry, and/or a Multi-Task Test. In certain embodiments, patient is dosed with 100 μg to 400 μg cebranopadol, as calculated based on equivalence to free base cebranopadol. In certain embodiments, the patient is dosed with greater than 450 μg to about 1000 μg cebranopadol, as calculated based on equivalence to free base cebranopadol. In certain embodiments, the dose is about 600 μg to about 1000 μg cebranopadol, as calculated based on equivalence to free base cebranopadol. In certain embodiments, the composition comprising the cebranopadol is a tablet unit dosage form. In certain embodiments, the tablet is a film coated tablet. In certain embodiments, the cebranopadol is in free base form. In certain embodiments, at least 80% of the cebranopadol is crystal form A.

In certain embodiments, a method is provided for reducing the abuse potential and side effects of Class II, III, and Class IV-opioids over a period of about 8 to 24 hours in a patient. The method comprises dosing a patient once a day with an immediate release composition comprising cebranopadol or a pharmaceutically acceptable salt thereof which has a mechanism of action comprising a dual receptor for nociceptin/orphanin FQ peptide (NOP) and μ-opioid peptide (MOP) receptor. In certain embodiments, the abuse potential is assessed using a visual analog scale (VAS) rating. Optionally, the abuse potential is further assessed using pupillometry and/or a Multi-Task Test. In certain embodiments, the method further comprises dosing the patient with cebranopadol at a supratherapeutic dose. A supratherapeutic dose may comprise greater than 450 μg to about 1000 μg cebranopadol, as calculated based on equivalence to free base cebranopadol. In certain embodiments, the supratherapeutic dose is about 600 μg to about 1000 μg cebranopadol, as calculated based on equivalence to free base cebranopadol. In certain embodiments, the composition comprises the cebranopadol is a tablet, optionally a film coated tablet.

In certain embodiments, a regimen is provided for reducing the abuse potential and sides effects of Class II and Class IV-opioids in a patient susceptible thereto. The method comprises: (a) discontinuing treatment of a patient with an opioid or opioid agonist which lacks a dual receptor for nociceptin/orphanin FQ peptide (NOP) and μ-opioid peptide (MOP) receptor; and (b) dosing a patient once a day with an immediate release composition comprising cebranopadol or a pharmaceutically acceptable salt thereof which comprises a dual receptor for nociceptin/orphanin FQ peptide (NOP) and μ-opioid peptide (MOP) receptor. In certain embodiments, (a) comprises titrating down the dosage of a Class II or Class IV opioid or opioid agonist by decreasing the dose of the opioid or opioid agonist in (a) over the period of 1 to 3 days. Steps (a) and (b) may be performed during the same or overlapping time periods. In certain embodiments, the opioid or opioid agonist of (a) is selected from tramadol, oxycodone, morphine, hydrocodone, fentanyl, oxymorphone, hydromorphone, buprenorphine, codeine, tapentadol, methadone, meperidine, or levorphanol. In certain embodiments, one or more of the side effects selected from nausea, vomiting, dizziness, pruritis, and/or hot flush sensation, are reduced or eliminated. In certain embodiments, the patient is renally or hepatically impaired.

In certain embodiments, a method is provided for treating pain in a patient having nociceptive pain with reduced risk of abuse. The regimen comprises dosing a patient once daily with an immediate release composition comprising cebranopadol or a pharmaceutically acceptable salt thereof. In certain embodiments, the composition used is cebranopadol free base. In certain embodiments, the composition is a film-coated tablet.

Still other aspects and advantages of the invention will be apparent from the following detailed description of the invention.

### DETAILED DESCRIPTION OF THE INVENTION

The present invention provides methods and compositions for treating pain using cebranopadol with opioid-level analgesic effect, but with much lower potential for abuse and negligible withdrawal and/or physical dependence compared to opioids, as well as significantly less respiratory depression as compared to opioids or opioid-like analgesics.

Without wishing to be bound by theory, the inventors have unexpectedly found that cebranopadol composition provides the activity of a full mu-agonist, while providing less abuse potential than a partial mu-agonist. The terms “μ” or “mu” are used interchangeably in reference to a full agonist or a partial agonist of the μ opioid receptor. Full agonists bind tightly to the opioid receptor and may undergo conformational changes to produce effect. Examples of full agonists may include, e.g., codeine, fentanyl, heroin, hydrocodone, hydromorphone, levorphanol, meperidine, methadone, morphine, oxycodone, and oxymorphone. These full agonists are typically classified as opioids as a Class (Schedule II) drug. In certain instances, a full agonist may be on Class (Schedule) III drug (e.g., codeine when mixed with acetaminophen). Examples of partial mu opioid receptor agonists includes tramadol and other opioid-like compounds, e.g., buprenorphine, butorphanol, typically classified as a Class (Schedule) IV drug.

It has surprisingly found that when cebranopadol is administered according to the methods provided herein, particularly crystal form A cebranopadol, subjects are therapeutically treated as effectively as with a full mu agonist, yet the potential for abuse is reduced in relation to partial mu opioids or opioid-like drugs, such as tramadol. This combination of properties is unexpected. In certain embodiments, the mu-agonist activity is assessed using a visual analog scale (VAS rating), pupillometry, and/or a Multi-Task Test.

Cebranopadol improves the rate of discontinuing taking the therapy at the end of the prescribed pain treatment as compared to known Schedule II, Class III, and class IV opioids and opioid-like analgesics. Cebranopadol provides analgesic effect comparable to a potent agonist such as fentanyl, while providing fewer treatment-related adverse events than comparable full agonists. In certain embodiments, a composition comprising cebranopadol treats a subject by delivering full mu (u) agonist activity (e.g., analgesic or other therapeutic effect), while avoiding addictive properties of full mu agonists (e.g., fentanyl, oxycodone, morphine, heroin, codeine, meperidine, or other Class I or Class III analgesics) and providing less addictive properties and/or less abuse potential than a partial mu agonist (e.g., tramadol or another Class IV opioid-like analgesic).

In further embodiments, unlike after treatment with oxycodone and tramadol, administration of cebranopadol does not produce pruritus, hyperhidrosis, feeling hot and/or hot flushing, that have been associated with the use of opioid analgesics. Provided herein are methods for providing a patient with opioid-level analgesic effect while preventing pruritus by administering cebranopadol. In certain embodiments, methods are provided for preventing pruritus in a patient receiving an opioid-level analgesic effect, comprising administering an effective amount of cebranopadol. Provided herein are methods for providing a patient with opioid-level analgesic effect while preventing hyperhidrosis by administering an effective amount of cebranopadol. In certain embodiments, methods are provided for preventing hyperhidrosis in a patient receiving an opioid-level analgesic effect, comprising administering an effective amount of cebranopadol. Provided herein are methods for providing a patient with opioid-level analgesic effect while preventing feeling hot and/or hot flushing by administering an effective amount of cebranopadol. In certain embodiments, methods are provided for preventing hot flushing in a patient receiving an opioid-level analgesic effect, comprising administering an effective amount of cebranopadol.

For the purpose of the specification, “treatment of pain” refers to any amelioration of pain, alleviation of pain or pain relief including the prevention thereof.

As used herein, “Cebranopadol” is intended to include trans-6′-fluoro-4′,9′-dihydro-N,N-dimethyl-4-phenyl-spiro[cyclohexane-1,1′-(3′H)-pyrano[3,4-b]indol]-4-amine (also referred to as (1r,4r)-6′-fluoro-N,N-dimethyl-4-phenyl-4′,9′-dihydro-3′H-spiro[cyclohexane-1,1′-pyrano[3,4-b]indol]-4-amine; free base: CAS Number 86351391-1), its pharmaceutically acceptable salts and solvates thereof: See, e.g., U.S. Pat. No. 7,799,931, incorporated by reference herein. See, also, crystal forms described in U.S. Pat. Nos. 8,895,604; 8,765,800, 8,618,156, and 8,614,245, which are incorporated herein by reference.

In certain embodiments, a free base form of cebranopadol is selected. In certain embodiments, a cebranopadol API composition comprises at least 50% to 100% of crystal form A, or at least 55%, at least 60%, at least 65%, at least 70%, at least 75%, at least 80%, at least 85%, at least 90%, at least 95%, at least 97%, at least 99%, or about 100% crystal form A. The crystal form may be present in a pharmaceutically acceptable salt form, e.g., HCl salt, and/or a hemihydrate, hydrate, solute, or anhydrous form. In certain embodiments, the cebranopadol is present in the active pharmaceutical ingredient (API) and/or the pharmaceutical composition as a free base and/or in crystal form A. See, Examples, Part A, incorporated herein by reference herein for the powder x-ray diffraction (PXRD) pattern for cebranopadol crystal form A. In certain embodiments, the cebranopadol crystal form A is characterized by one or more of the following: the PXRD pattern of the Table in Part A,

X-ray powder diffraction pattern comprising characteristic peaks at 7.8±0.2 degrees 2θ and at 31.6±0.2 degrees 2θ, and wherein the active ingredient comprises (1r,4r)-6′-fluoro-N,N-dimethyl-4-phe-nyl-4′,9′-dihydro-3, EI-spiro-[cyclohexane-1,1,-pyrano-[3,4, b]indol]-4-amine at a diasteromeric excess of about 90% de. In certain embodiments, the crystalline form comprises a characteristic peak at 11.7±0.2 degrees 2θ. In certain embodiments, the crystalline form comprises characteristic peak at 18.3±0.2 degrees 2θ. In certain embodiments, the crystalline form comprises characteristic peaks at 8.8±0.2 degrees 2θ and/or at 15.8±0.2 degrees 2θ. In certain embodiments, the crystalline form comprises characteristic peaks at about 20.4±0.2 degrees 2θ and/or at 23.3±0.2 degrees 2θ. In certain embodiments, the crystalline form comprises characteristic peaks at 11.7±0.2 degrees 2θ, at one or both of 8.8±0.2 degrees 2θ and/or 15.8±0.2 degrees 2θ, and at one or both of 20.4±0.2 degrees 2θ and/or 23.3±0.2 degrees 2θ. In certain embodiments, the crystalline form has an endothermal event with a peak temperature at about 298-308° C., as determined by DSC. In certain embodiments, the crystalline form A has a Raman peak at about 1569±2 cm-1 and/or at about 1002±2 cm-1. In certain embodiments, the active ingredient comprises a (1r,4r)-6′-fluoro-N,N-dim-ethyl-4-phenyl-4′,9,-dihydro-3,H-spiro-[cyclohexane-1,T-pyrano-[3,4,b]indol]-4-amine at a diasteromeric excess of at least about 95% de. In certain embodiments, the active ingredient comprises a (1r,4r)-6′-fluoro-N,N-dimethyl-4-phenyl-4′,9,-dihydro-3,H-spiro-[cyclohexane-1, T-pyrano-[3,4,b]indol]-4-amine at a diasteromeric excess of at least about 97% de. In certain embodiments, the active ingredient comprises a (1r,4r)-6′-fluoro-N,N-dim-ethyl-4-phenyl-4′,9,-dihydro-3,H-spiro-[cyclohexane-1,T-pyrano-[3,4,b]indol]-4-amine at a diasteromeric excess of at least about 99% de. In certain embodiments, crystalline form A is present in the active ingredient in an amount of at least about 60 wt. % relative to the total weight of all crystalline and non-crystalline forms of (1r,4r-6′-fluoro-N,N-dimethyl-4-phenyl-4′,9′-dihydro-3,H-spiro-[cyclohex-ane-1, T-pyrano-[3,4,b]indol]-4-amine. In certain embodiments, crystalline form A is present in the active ingredient in an amount of at least about 80 wt. % relative to the total weight of all crystalline and non-crystalline forms of (1r,4r-6′-fluoro-N,N-dimethyl-4-phenyl-4′,9′-dihydro-3,H-spiro-[cyclohex-ane-1, T-pyrano-[3,4,b]indol]-4-amine. In certain embodiments, crystalline form A is present in the active ingredient in an amount of at least about 90 wt. % relative to the total weight of all crystalline and non-crystalline forms of (1r,4r-6′-fluoro-N,N-dimethyl-4-phenyl-4′,9′-dihydro-3,H-spiro-[cyclohex-ane-1, T-pyrano-[3,4,b]indol]-4-amine. In certain embodiments, crystalline form A is present in the active ingredient in an amount of at least about 95 wt. % relative to the total weight of all crystalline and non-crystalline forms of (1r,4r)-6′-fluoro-N,N-dimethyl-4-phenyl-4′,9′-dihydro-3,H-spiro-[cy-clohexane-1,l′-pyrano-[3,4,b]indol]-4-amine. In certain embodiments, the pharmaceutical composition contains at most about 1.0 wt.-% 4-dimethylamino-4-phenylcyclohexanone, relative to the total content of 6′-fluoro-N,N-dimethyl-4-phenyl-4′,9′-dihydro-3′H-spiro[cyclohexane-1,T-pyran [3,4b]indol]-4-amine. In certain embodiments, the composition contains at most about 1.0 wt.-% 4-dimethylamino-4-phenylcyclohexanone, relative to the total content of 6′-fluoro-N,N-dimethyl-4-phenyl-4′,9′-dihydro-3′H-spiro[cyclohexane-1, T-pyran [3,4b]indol]-4-amine.

Methods of making the compound are described, e.g., U.S. Pat. Nos. 8,779,160; 8,658,827; 10,323,040, all of which are incorporated by reference herein.

Although the free base of cebranopadol is preferred, one may select a pharmaceutically acceptable salts of Cebranopadol, which may include salts of inorganic acids, such as hydrochloric acid (Cebranopadol HCl), hydrobromic acid and sulfuric acid, and salts of organic acids, such as methane sulfonic acid, fumaric acid, maleic acid, acetic acid, oxalic acid, succinic acid, malic acid, tartaric acid, mandelic acid, lactic acid, citric acid, glutamic acid, acetylsalicylic acid, nicotinic acid, aminobenzoic acid, α-liponic acid, hippuric acid and asparaginic acid. In certain embodiments, Cebranopadol is present in the non-salt form (free base). In other embodiments, the cebranopadol is present as cebranopadol hemicitrate (CAS number CAS No. 863513-92-2).

In certain embodiments, a cebranopadol compound useful in certain embodiments of the invention has the structure of:

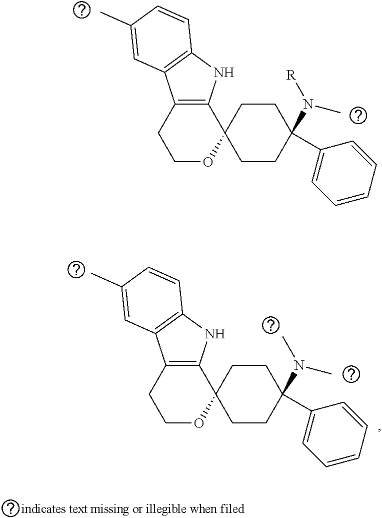

Cebranopadol's duration of action following an immediate release dose is long, e.g., up to 7 hours after intravenous dosing (e.g., 5 to 7 hours), or greater than 9 hours after oral dosing (e.g, 8 to 16 hours, 9 to 18 hours, or longer, e.g., 8 to 24 hours.

For the purpose of the specification, doses of Cebranopadol relate to the free base.

As used herein, “micronized” cebranopadol refers to the size of the drug particle, in which the average size of the drug particles are less than 10 microns.

Thus, when a pharmaceutically acceptable salt is used instead, its dose has to be adapted to the equivalent dose of the free base. For example, a dose of “200 μg” means an amount of 200 μg of the free base or any equivalent amount of a pharmaceutically acceptable salt or solvate corresponding to 200 μg of the free base.

Provided herein are unit dosage forms, e.g., coated tablets, comprising 100 μg cebranopadol, 200 μg cebranopadol, 300 μg cebranopadol, or 400 μg cebranopadol, wherein the dose is determined based on equivalence to the free base. In certain embodiments, these unit dosage forms comprise micronized cebranopadol free base as the sole active ingredient. In other embodiments, other forms of cebranopadol are present in the unit dosage form.

Cebranopadol or the physiologically acceptable salt thereof may be administered systemically or orally. In certain embodiments, Cebranopadol or the physiologically acceptable salt thereof is administered once daily.

While the examples provided herein were generated with one illustrative composition comprising the cebranopadol, other compositions may be used in the methods provided herein. See, e.g., U.S. Pat. No. 9,289,416, which is incorporated by reference herein.

As used herein, the term micrograms is abbreviated “ug” or “mcg”, which may be used interchangeably.

As used herein, for an adult patient experiencing acute pain, a supratherapeutic dose generally involves delivery of a dose in excess of 400 μg to 2000 μg, e.g., at least 450 μg, at least 500 μg, at least 550 μg, at least 600 μg, at least 650 μg, at least 700 μg, at least 750 μg, at least 800 μg, at least 850 μg, at least 900 μg, at least 950 μg, at least 1000 μg, at least 1100 μg, at least 1200 μg, at least 1300 μg, at least 1400 μg, at least 1500 μg, at least 1600 μg, at least 1700 μg, at least 1800 μg, at least 1900 μg, or at least 2000 μg, as equivalent dose relative to Cebranopadol free base. In certain embodiments, the supratherapeutic dose is at least 600 μg, at least 450 μg, at least 500μ, at least 550μ, at least 600μ, at least 650μ, at least 700 μg, at least 750 μg, at least 800 μg, at least 850 μg, at least 900 μg, at least 950 μg, at least 1000 μg, at least 1100 μg, at least 1200 μg, at least 1300 μg, at least 1400 μg, at least 1500 μg, at least 1600 μg, at least 1700 ug, at least 1800 μg, at least 1900 μg, or at least 2000 μg, as equivalent dose relative to Cebranopadol free base.

Doses may be titrated, e.g., as described in U.S. Pat. No. 10,022,353 which is incorporated herein by reference, using a subtherapeutic analgesic dose(s) as the starting dose. In certain embodiment, the cebranopadol subtherapeutic analgesic doses are combined into a therapeutic regimen comprising a dosing regimen which comprises starting at a subtherapeutic dose or a therapeutic dose of cebranopadol, and incorporating a supratherapeutic dose or doses into the otherwise titrated regimen over 1-3 weeks, or as needed. In certain embodiments, a patient may receive a supratherapeutic dose for 1, 3 or 3 days, followed by therapeutic or subtherapeutic doses on subsequent days. In certain embodiments, cebranopadol may be delivered in a regimen comprising a single daily dosage delivered over a period of days to weeks without a change in daily dosage. Alternatively, cebranopadol may be delivered in a first dose, followed by an increase in daily dose on day 2 and subsequent days. Alternatively dosage is adjusted as needed.

The duration of treatment is not particularly limited and may last for several weeks, months, or years, especially when the pain to be treated or prevented is chronic. In certain embodiments, when the pain is chronic, the pain is treated for at least one week or at least two weeks.

Pain and/or opioid drug dependence are treated or prevented. When pain is to be treated or prevented, the pain may be moderate, moderate to severe, or severe. The pain may be chronic or acute; and/or central and/or peripheral; and/or neuropathic and/or nociceptive. In connection with central/peripheral pain and with nociceptive/neuropathic pain “and/or” reflects the possibility that the overall pain may have different components, e.g., a nociceptive component as well as a neuropathic component. In certain embodiments, the pain is chronic neuropathic pain, which may be peripheral or central; acute neuropathic pain, which may be peripheral or central; chronic nociceptive pain, which may be peripheral or central; or acute nociceptive pain, which may be peripheral or central.

Nociceptive pain refers to the discomfort that results when a stimulus causes tissue damage to the muscles, bones, skin or internal organs. For the purpose of the specification, nociceptive pain is caused by stimulation of peripheral nerve fibers that respond only to stimuli approaching or exceeding harmful intensity (nociceptors), and may be classified according to the mode of noxious stimulation; the most common categories being “thermal” (heat or cold), “mechanical” (crushing, tearing, etc.) and “chemical” (iodine in a cut, chili powder in the eyes).

Nociceptive pain may also be divided into “visceral,” “deep somatic” and “superficial somatic” pain.

Visceral pain describes a type of nociceptive pain originating in the body's internal organs or their surrounding tissues. This form of pain usually results from the infiltration of harmful cells, as well as the compression or extension of healthy cells. Subjects suffering from visceral pain tend to feel generally achy, as this pain tends to not be localized to a specific area. Cancer is a common source of visceral pain.

Somatic pain is nociceptive pain that results from some injury to the body. It's generally localized to the affected area and abates when the body repairs the damage to that area. Deep somatic pain is initiated by stimulation of nociceptors in ligaments, tendons, bones, blood vessels, fasciae and muscles, and is dull, aching, poorly localized pain. Examples include sprains and broken bones. Superficial pain is initiated by activation of nociceptors in the skin or superficial tissues, and is sharp, well-defined and clearly located.

Pain may be classified as chronic if it has occurred for at least 3 months or extends beyond the time of healing. In certain embodiments, the chronic nociceptive pain is selected from chronic visceral pain, chronic deep somatic pain and chronic superficial somatic pain.

Causes of nociceptive pain include broken or fractured bones, bruises, burns, cuts, inflammation (from infection or arthritis), and sprains. Thus, nociceptive pain includes postoperative pain, cancer pain, low back pain, pain due to radiculopathy, and inflammatory pain. Neuropathic pain is pain that originates from nerve damage or nerve malfunction. In certain embodiments, the neuropathic pain is selected from acute neuropathic pain and chronic neuropathic pain. Neuropathic pain may be caused by damage or disease affecting the central or peripheral portions of the nervous system involved in bodily feelings (the somatosensory system). In certain embodiments, the composition is for use in the treatment of chronic neuropathic pain or acute neuropathic pain, peripheral neuropathic pain or central neuropathic pain, mononeuropathic pain or polyneuropathie pain. When the neuropathic pain is chronic, it may be chronic peripheral neuropathic pain or chronic central neuropathic pain, in certain embodiments, chronic peripheral mononcuropathic pain or chronic central mononeuropathic pain, in certain embodiments, chronic peripheral polyneuropathie pain or chronic central polyneuropathie pain. When the neuropathic pain is acute, it may be acute peripheral neuropathic pain or acute central neuropathic pain, in certain embodiments, acute peripheral mononeuropathic pain or acute central mononeuropathic pain, in certain embodiments, acute peripheral polyneuropathie pain or acute central polyneuropathie pain.

Central neuropathic pain is found in spinal cord injury, multiple sclerosis, and some strokes. Fibromyalgia is potentially a central pain disorder and is responsive to medications that are effective for neuropathic pain. Aside from diabetic neuropathy and other metabolic conditions, the common causes of painful peripheral neuropathies are herpes zoster infection, HIV-related neuropathies, nutritional deficiencies, toxins, remote manifestations of malignancies, genetic, and immune mediated disorders or physical trauma to a nerve trunk (e.g., due to disorders from the spinal disc, joint degeneration, or compression fracture). Neuropathic pain is common in cancer as a direct result of cancer on peripheral nerves (e.g., compression by a tumor), or as a side effect of chemotherapy, radiation injury or surgery.

In certain embodiments, the pain is selected from postoperative pain, pain due to bunionectomy, visceral pain, cancer pain, pain due to diabetic polyneuropathy, pain due to osteoarthritis, fibromyalgia, low back pain, pain radiating down the lower limbs, pain due to (cervical or lumbar) radiculopathy, and inflammatory pain.

In certain embodiments, the pain is selected from the group consisting of pain being or being associated with panic disorder [episodic paroxysmal anxiety]; dissociative [conversion] disorders; persistent somatoform pain disorder; pain disorders exclusively related to psychological factors; nonorganic dyspareunia; other enduring personality changes; sadomasochism; elaboration of physical symptoms for psychological reasons; migraine; other headache syndromes; trigeminal neuralgia [G50.0]; atypical facial pain [G50.1]; phantom limb syndrome with pain [G54.6]; phantom limb syndrome without pain [G54.7]; acute and chronic pain, not elsewhere classified [G89]; ocular pain [H57.1]; otalgia [H92.0]; angina pectoris, unspecified [120.9]; other specified disorders of nose and nasal sinuses [J34.8]; other diseases of pharynx [J39.2]; temporomandibular joint disorders [K07.6]; other specified disorders of teeth and supporting structures [K08.8]; other specified diseases of jaws [K10.8]; other and unspecified lesions of oral mucosa [K13.7]; glossodynia [K14.6]; other specified diseases of anus and rectum [K62.8]; pain in joint [M25.5]; shoulder pain [M25.51]; sacrococcygeal disorders, not elsewhere classified [M53.3]; spine pain [M54.]; radiculopathy [M54.1]; cervicalgia [M54.2]; sciatica [M54.3]; low back pain [M54.5]; pain in thoracic spine [M54.6]; other dorsalgia [M54.8]; dorsalgia, unspecified [M54.9]; other shoulder lesions [M75.8]; other soft tissue disorders, not elsewhere classified [M79]; myalgia [M79.1]; neuralgia and neuritis, unspecified [M79.2]; pain in limb [M79.6]; other specified disorders of bone [M89.8]; unspecified renal colic [N23]; other specified disorders of penis [N48.8]; other specified disorders of male genital organs [N50.8]; mastodynia [N64.4]; pain and other conditions associated with female genital organs and menstrual cycle [N94]; mittelschmerz [N94.0]; other specified conditions associated with female genital organs and menstrual cycle [N94.8]; pain in throat and chest [R07]; pain in throat [R07.0]; chest pain on breathing [R07.1]; precordial pain [R07.2]; other chest pain [R07.3]; chest pain, unspecified [R07.4]; abdominal and pelvic pain [R10]; acute abdomen pain [R10.0]; pain localized to upper abdomen [R10.1]; pelvic and perineal pain [R10.2]; pain localized to other parts of lower abdomen [R10.3]; other and unspecified abdominal pain [R10.4]; flatulence and related conditions [R14]; abdominal rigidity [R19.3]; other and unspecified disturbances of skin sensation [R20.8]; pain associated with micturition [R30]; other and unspecified symptoms and signs involving the urinary system [R39.8]; headache [R51]; pain, not elsewhere classified [R52]; acute pain [R52.0]; chronic intractable pain [R52.1]; other chronic pain [R52.2]; pain, unspecified [R52.9]; other complications of cardiac and vascular prosthetic devices, implants and grafts [T82.8]; other complications of genitourinary prosthetic devices, implants and grafts [T83.8]; other complications of internal orthopedic prosthetic devices, implants and grafts [T84.8]; other complications of internal prosthetic devices, implants and grafts, not elsewhere classified [T85.8]; wherein the information in brackets refers to the classification according to ICD-10.

Depending upon the type and degree of pain to be treated or prevented, Cebranopadol or the physiologically acceptable salt thereof is administered at a dose that in the subject's perception results in an amelioration of pain at acceptable side effects. Typically, the dose is within the range of from 20 μg to 2000 μg, as equivalent dose relative to Cebranopadol free base.

In certain embodiments, the dosage form is adapted for administration once daily and contains the pharmacologically active agent in a dose of from 150 μg to 800 μg, more than 190 μg to 800 μg, i.e., the dosage form contains the pharmacologically active agent (e.g, cebranopadol or salt thereof) in a daily dose of from 150 μg to 800 μg. In a certain embodiment, the dose is from 200 μg to 800 μg, from 210 μg to 750 μg, from 220 μg to 700 μg, from 230 μg to 650 μg, from 240 μg to 600 μg, from 250 μg to 550 μg. In certain embodiments, as Cebranopadol or the physiologically acceptable salt thereof is administered once daily, this dose corresponds to the daily dose.

For the purpose of the specification, “administration once daily” (sid, OD) in certain embodiments means that the pharmaceutical composition is adapted for being administered according to a regimen comprising the administration of a first pharmaceutical composition and the subsequent administration of a second pharmaceutical composition according to the invention, wherein both, the first and the second pharmaceutical composition are administered during a time interval of about 48 hours, but wherein the second pharmaceutical composition is administered not earlier than 18 hours, not earlier than 20 hours, not earlier than 22 hours and in particular, about 24 hours after the first pharmaceutical composition has been administered.

Administration regimens “once daily” may be realized by administering a single pharmaceutical composition containing the full amount of the cebranopadol or pharmaceutically acceptable salt thereof to be administered at a particular point in time or, alternatively, administering a multitude of dose units, i.e. two, three or more dose units, the sum of which multitude of dose units containing the full amount of the cebranopadol or a pharmaceutically acceptable salt thereof to be dosed at said particular point in time, where the individual dose units are adapted for simultaneous administration or administration within a short period of time, e.g. within 5, 10 or 15 minutes.

In certain embodiments, a pharmaceutical composition (e.g., pharmaceutical dosage form) comprises at least one form of cebranopadol and/or a pharmaceutically acceptable salt thereof, or a hydrate of the cebranopadol or salt thereof, or a solvate of a cebranopadol or a salt or hydrate thereof. In certain embodiments, the pharmaceutical composition provides immediate release of the cebranopadol or pharmaceutically acceptable salt thereof (or other active ingredient). Such a pharmaceutical composition may be specifically designed to provide immediate release of the cebranopadol in accordance with Ph. Eur or the equivalent. When the pharmaceutical composition is coated, e.g., with a coating that is soluble in gastric juice, the release kinetic may be monitored after such coating has been dissolved.

For the purpose of specification, the term “immediate release” refers to any release profile that fulfills at least one, preferably both, of the following requirements. First, the pharmaceutical composition disintegrates in 10 minutes or less following exposure to a disintegrating medium. Methods to determine the disintegration time are known to a person skilled in the art. For instance, they can be determined according to the USP XXIV disintegration test procedure, using, for example, an Erweka ZT-71 disintegration tester. Second, the pharmaceutical composition releases at least 70 wt % of the drug within 15 minutes following exposure to a dissolution medium. In certain embodiments, the in vitro release properties of the pharmaceutical composition (dosage form) are determined according to the paddle method with sinker at 50, 75 or 100 rpm, under in vitro conditions at 37±0.5° C. in 900 mL artificial gastric juice at pH 1.2, or under the same conditions in non-artificial gastric juice. In certain embodiments, the pharmaceutical composition releases under in vitro conditions in 900 mL artificial gastric juice at pH 1.2 and 37±0.5° C. after 30 minutes according to the paddle method with sinker at 100 rpm at least 50 wt %, at least 60 wt %, at least 70 wt %, at least 80 wt %, at least 90 wt %, or at least 95 wt % of the cebranopadol or salt thereof, based on the total amount of the cebranopadol or salt thereof originally contained in the pharmaceutical composition.

Various components for pharmaceutical compositions may be selected. See, e.g., U.S. Pat. Nos. 9,289,416 and 10,912,763, which are incorporated by reference herein. In certain embodiments, the pharmaceutical composition is a tablet, chewable tablet, chewing gum, coated tablet or powder, optionally filled into a capsule. In certain embodiments, the pharmaceutical composition in multi-particulate form, in form of a micro-tablet, micro capsule, granulate, pellet or active-substance crystal, optionally filled into a capsule or compressed to form a tablet. A solid pharmaceutical composition may contain pharmaceutical excipients including one or more lubricants, binders, disintegrants, fillers, diluents, glidants, surfactants, and preservatives.

One suitable lubricant that may be contained in the pharmaceutical composition is magnesium stearate, the content of the lubricant is within the range of from 0.001 to 5.0 wt %, for example 0.01 to 5 wt.-%, 0.1 to 5 wt.-%, 0.1 to 3 wt.-%, 0.1 to 2 wt.-%, or even 0.5 to 1.5 wt.-%, based on the total weight of the composition (e.g., tablet). In certain embodiments, the pharmaceutical composition further contains a binder. Suitable binders include but are not limited to gelatin, cellulose, modified cellulose such as microcrystalline cellulose, methyl cellulose, polyvinyl pyrrolidone (povidone), starch, sucrose and polyethylene glycol; especially preferred are polyvinyl pyrrolidone and/or microcrystalline cellulose. In certain embodiments, the content of lubricant is within the range of from 0.001 to 30 wt.-%, or 0.1 to 25 wt.-%, based on the total weight of the composition (e.g., tablet). In some embodiments, the composition comprises 1 to 20 wt.-%, 5 to 20 wt.-%, or 10 to 20 wt.-% of binder(s), based on the total weight of the composition (e.g., tablet). In certain embodiments, the pharmaceutical composition further contains a filler and/or diluent, e.g., selected from the group consisting of but are not limited to cellulose (e.g., microcrystalline cellulose), calcium diphosphate, lactose (e.g., lactose monohydrate), sucrose, glucose, mannitol, sorbitol, and calcium carbonate. In certain embodiments, the content of filler and/or diluent is within the range of from 0.001 to 95 wt.-%, 30 wt % to about 90 wt %, 0.01 to 85 wt.-%, 0.1 to 80 wt.-%, or 10 to 75 wt.-%, based on the total weight of the composition (e.g., tablet). In certain embodiments, the pharmaceutical composition further contains a lubricant such as magnesium stearate, stearic acid and stearin. In certain embodiments, the content of the lubricant is within the range of from 0.001 to 5 wt %, e.g., from 0.1 to 3 wt %, or about 0.5 wt % to 1.5% wt %, based on the total weight of the composition (e.g., tablet). In certain embodiments, the pharmaceutical composition further contains a disintegrant such as cross-linked sodium carboxymethyl cellulose (croscarmellose sodium), cross-linked polyvinyl pyrrolidone and sodium starch glycolate. In certain embodiments, the content of the disintegrant is within the range of from 0.001 to 5 wt. %, e.g., from 0.1 to 3 wt. %, based on the total weight of the composition (e.g., tablet). The pharmaceutical composition may further contain at least one preservative. Suitable preservatives include but are not limited to antioxidants, such as vitamin A, vitamin E, vitamin C, retinyl palmitate and selenium; cysteine, methionine, citric acid, sodium citrate, methyl paraben and propyl paraben.

In certain embodiments, a solid pharmaceutical composition further contains a coating, in particular a polymer-based coating, more in particular a polyvinyl alcohol-based coating such as the ones commercially available under the trade name “Opadry”. In some embodiments, the pharmaceutical composition is a tablet which comprises the cebranopadol or pharmaceutically acceptable salt thereof (e.g., in an amount from 0.6±0.4 wt %, 0.6-0.3 wt-%, 0.6±0.2 wt %, 0.6±0.1 wt %, 0.04±0.03 wt %, 0.04±0.02 wt. %, or 0.04±0.01 wt %), one or more lubricants (e.g., magnesium stearate) in an amount from 0.001 to 5.0 wt. % (e.g., 0.01 to 5 wt %, 0.1 to 5 wt %, 0.1 to 3 wt %, 0.1 to 2 wt %, or even 0.5 to 1.5 wt %), one a more binders (e.g., polyvinyl pyrrolidone and/or microcrystalline cellulose) in an amount from 0.001 to 30 wt % (e.g., from 0.1 to 25 wt %, 1 to 20 wt %, 5 to 20 wt %, or 10 to 20 wt %), and one or more fillers or diluents (e.g., microcrystalline cellulose and/or lactose) in an amount from 0.001 to 90 wt % (e.g., 0.01 to 85 wt %, 0.1 to 80 wt %, or 10 to 75 wt %), based on the total weight of the composition (e.g., tablet). In some embodiments, the tablet also comprises one or more lubricants (e.g., magnesium stearate, stearic acid and/or stearin) in an amount from 0.001 to 5 wt % (e.g., from 0.1 to 3 wt %) and/or one or more disintegrants (e.g., croscarmellose sodium, cross-linked polyvinyl pyrrolidone and/or sodium starch glycolate) in an amount from 0.001 to 5 wt % (e.g., from 0.1 to 3 wt %), based on the total weight of the composition (e.g., tablet). In certain embodiments, the coating protects the pharmaceutical composition from moisture, but dissolves rapidly in gastric juice. In certain embodiments, the coated composition has a disintegration time of less than 5 minutes in gastric juice, of at most 4.5 minutes, at most 4 minutes, at most 3.5 minutes, at most 3 minutes, at most 2.5 minutes and/or at most 2 minutes. For the manufacture of the pharmaceutical compositions, the various solid auxiliary substances and the pharmacologically active agent may be homogenized, processed by means of wet, dry or fusion granulation to form granulates, and compressed to form tablets. Alternatively, they are manufactured by direct tableting of the auxiliary substances and the pharmacologically active agent. In certain embodiments, the pharmaceutical composition is prepared by means of wet granulation from a granulating fluid containing the pharmacologically active agent in particular from an aqueous granulating fluid containing said pharmacologically active agent and the surfactant. In certain embodiments, the resulting granulating fluid is then top-sprayed or bottom-sprayed onto a solid formulation containing at least one auxiliary substance to yield compressible granules, which may optionally be mixed with further auxiliary substances before being compressed to tablets.

Further provided herein are methods and regimens using the pharmaceutical compositions comprising at least cebranopadol or a pharmaceutically acceptable salt, hydrate or solvate thereof. In certain embodiments, the composition comprises cebranopadol free base. In certain embodiments, the composition is an immediate release composition.

In certain embodiments, a method is provided for reducing the abuse potential and side effects of Class II, III and Class IV-opioids over a period of about 8 to 24 hours in a patient. The method comprises dosing a patient once a day with an immediate release composition comprising cebranopadol or a pharmaceutically acceptable salt thereof which has a mechanism of action comprising a dual receptor for nociceptin/orphanin FQ peptide (NOP) and μ-opioid peptide (MOP) receptor. In certain embodiments, the abuse potential is assessed using a visual analog scale (VAS) rating. Optionally, the abuse potential is further assessed using pupillometry and/or a Multi-Task Test. In certain embodiments, the method further comprises dosing the patient with cebranopadol at a dose of about 50 μg to 800 μg or more than 190 μg to 800 μg, i.e., the dosage form a contains the pharmacologically active agent in a daily dose of from 150 μg to 800 μg. In certain embodiments, the dose of the pharmacologically active agent is in the range of from 200 μg to 800 μg, from 210 μg to 750 μg, from 220 μg to 700 μg, from 230 μg to 650 μg, from 240 μg to 600 μg, from 250 μg to 550 μg. In certain embodiments, a supratherapeutic dose may be selected. A supratherapeutic dose may comprise greater than 450 μg to about 1000 μg cebranopadol, as calculated based on equivalence to free base cebranopadol. In certain embodiments, the supratherapeutic dose is about 600 μg to about 1000 μg cebranopadol, as calculated based on equivalence to free base cebranopadol. In certain embodiments, the composition comprises the cebranopadol is a tablet, optionally a film coated tablet.

In certain embodiments, a regimen is provided for reducing the abuse potential and sides effects of Class II and Class IV-opioids in a patient susceptible thereto. The method comprises: (a) discontinuing treatment of a patient with an opioid or opioid agonist which lacks a dual receptor for nociceptin/orphanin FQ peptide (NOP) and μ-opioid peptide (MOP) receptor; and (b) dosing a patient once a day with an immediate release composition comprising cebranopadol or a pharmaceutically acceptable salt thereof which comprises a dual receptor for nociceptin/orphanin FQ peptide (NOP) and μ-opioid peptide (MOP) receptor. In certain embodiments, (a) comprises titrating down the dosage of a Class II or Class IV opioid or opioid agonist by decreasing the dose of the opioid or opioid agonist in (a) over the period of 1 to 3 days. Steps (a) and (b) may be performed during the same or overlapping time periods. In certain embodiments, the opioid or opioid agonist of (a) is selected from tramadol, oxycodone, morphine, hydrocodone, fentanyl, oxymorphone, hydromorphone, buprenorphine, codeine, tapentadol, methadone, meperidine, or levorphanol. In certain embodiments, one or more of the side effects selected from nausea, vomiting, dizziness, pruritis, and/or hot flush sensation, are reduced or eliminated. In certain embodiments, the patient is renally or hepatically impaired.

In certain embodiments, a method is provided for treating pain in a patient having nociceptive pain with reduced risk of abuse.

In certain embodiments, a regimen comprises dosing a patient once daily with an immediate release composition comprising cebranopadol or a pharmaceutically acceptable salt thereof. In certain embodiments, the composition used is cebranopadol free base. In certain embodiments, the composition is a film-coated tablet. In certain embodiments, a dosage unit comprises 100 mcg (μg) cebranopadol (equivalent to free base). In certain embodiments, a dosage unit comprises 200 meg cebranopadol (equivalent to free base). In certain embodiments, a dosage unit is 300 mcg cebranopadol (equivalent to free base). In certain embodiments, a dosage unit is 400 mcg cebranopadol. In certain embodiments, a single daily oral dose may comprise 1, 2, 3, 4, 5, 6, 7, or 8 tablets taken essentially at the same time (e.g., 100 mcg tablet taken within five minutes of each other). In certain embodiments, a single daily oral dose may comprise 1, 2, 3 or 4 tablets (e.g., 200 mcg tablet taken within five minutes of each other). In certain embodiments, a single daily oral dose may comprise 1 or 2 tablets (e.g., 100, 200, 300 or 400 mcg tablets, or combinations thereof, taken within five minutes of each other). Other combinations may be selected.

The pharmacokinetic parameters of cebranopadol may be calculated from plasma concentration-time data.

AUC₀₋ₜ refers to “Area under the concentration-time curve” from administration up to the sampling time t. If it is not replaced by a numerical value, then t is the last sampling time with quantifiable concentration and this parameter will not contain an extrapolated portion.

AUC_{inf} refers to Area under the concentration-time curve from time 0 to infinite time.

Areas under the curves in the study examples below may be calculated using the log-linear trapezoidal rule, i.e., linear up to the maximum concentration and log thereafter. However, others may readily select another method.

Cₘₐₓ refers to the maximum observed plasma concentration level.

Tₘₐₓ refers to the time post-dosing to attain maximum plasma concentration.

The PK values may be determined using geometric mean and/or the arithmetic mean. In certain embodiments, mean, individual, and overlay concentration-time profiles are plotted on both linear and semi-logarithmic scales on the same, portrait-oriented page.

Pharmacodynamics (PD) may be assessed using a Visual Analog Scale (VAS), Pupillometry, and/or the Multi-Tasking Test (MTT), such as described in the examples herein. Additional or alternative tests may be selected.

The VAS assessment is one of the most sensitive indices of abuse liability. See, Babalonis S, Lofwall M R, Nuzzo P A, Siegel A J, Walsh S L. Abuse liability and reinforcing efficacy of oral tramadol in humans. Drug Alcohol Depend. 2013 Apr. 1; 129 (1-2): 116-241; Food and Drug Administration Guidance for Industry. Assessment of Abuse Potential of Drugs. January 2017.] The VAS for Drug Liking assesses the subject's liking of the drug at this moment the question is asked. The VAS is a bipolar scale. The scale is not administered pre-dose as it refers specifically to the drug.

Pupillometry may be used as an objective physiological PD measure as it is one of the most sensitive measures of central opioid action and appears to be resistant to tolerance development with repeated opioid administration. NeurOptics Pupillometer (Irvine, CA, USA) or similar equipment will be used to measure pupil diameter. Data from a series of frames will be used in the calculation, and the final display will show the weighted average and standard deviation of the pupil size. Measurements may be collected under mesopic lighting conditions.

The Multi-Tasking Test (MTT) (formerly known as the Attention Switching Task, AST) is a test of executive function which provides a measure of the ability to use multiple sources of potentially conflicting information to guide behavior. In this task the participant is presented with a series of arrows on-screen, pointing in either direction (to the right or to the left). Each trial displays a cue at the top of the screen that indicates to the participant whether they have to press the right or left button according to the “side on which the arrow appeared” or the “direction in which the arrow was pointing”. Some trials display congruent stimuli (e.g., arrow on the right side of the screen pointing to the right) whereas other trials display incongruent stimuli which require a higher cognitive demand (e.g., arrow on the right side of the screen pointing to the left). In the final section, both rules are used, presented in a randomized order, requiring the participant to adjust their response depending on whether the rule is repeated or switched (multitasking). Outcome measures for the Multitasking Test include response latencies and error scores that reflect the participant's ability to manage multitasking and the interference of incongruent task-irrelevant information on task performance. In certain embodiments, the administration time for the test is about 8 minutes.

In certain embodiments, a cebranopadol or a pharmaceutically acceptable salt, hydrate, or salt hydrate, is provided which when administered to a subject provides the subject with a therapeutic effect of a pharmaceutically acceptable full mu agonist and a lower potential for abuse than a pharmaceutically acceptable partial mu agonist. In certain embodiments, the subject is being treated for pain. In certain embodiments, the pain is chronic acute; central; peripheral; neuropathic and/or nociceptive pain, or another of the types of pain provided in this specification or known in the art. In certain embodiments, the dose is administered daily in an amount of about 10 μg to about 2000 μg cebranopadol. In certain embodiments, the cebranopadol is a free base. In certain embodiments, at least 80% of the cebranopadol is in crystal form A. In certain embodiments, the partial mu agonist is tramadol.

In certain embodiments, use of cebranopadol in treating a subject is provided, wherein the composition comprises cebranopadol or a pharmaceutically acceptable salt, hydrate, or salt hydrate, which when administered to a subject provides the subject with a full mu agonist therapeutic effect and a lower abuse potential than a partial mu agonist. In certain embodiments, use of cebranopadol in preparing a medicament for treating a subject is provided, wherein the composition comprises cebranopadol or a pharmaceutically acceptable salt, hydrate, or salt hydrate, which when administered to a subject provides the subject with a full mu agonist therapeutic effect and a lower abuse potential than a partial mu agonist.

In certain embodiments, a method, use or composition is provided for reducing the abuse potential and sides effects of Class II, III and Class IV-opioids and opioid-like analgesics over a period of about 8 to 24 hours in a patient in need of analgesic treatment, said method comprising dosing a patient once a day with an immediate release composition comprising cebranopadol or a pharmaceutically acceptable salt or hydrate thereof. In certain embodiments, the mu-agonist effect is assessed using a visual analog scale (VAS) rating, pupillometry and/or a Multi-Task Test. In certain embodiments, the patient is dosed with 100 μg to 400 μg cebranopadol, as calculated based on equivalence to free base cebranopadol. In certain embodiments, the cebranopadol is at dose which comprises greater than 450 μg to about 1000 μg cebranopadol, or about 600 μg to about 1000 μg, as calculated based on equivalence to free base cebranopadol. In certain embodiments, the cebranopadol is a tablet unit dosage form. In certain embodiments, the tablet is a film coated tablet. In certain embodiments, the cebranopadol is in free base form. In certain embodiments, at least 80% of the cebranopadol is crystal form A.

In certain embodiments, a regimen is provided for reducing the abuse potential and sides effects of Class II and Class IV-opioids in a patient susceptible thereto, said method comprising: (a) discontinuing treatment of a patient with an opioid or opioid agonist which lacks a dual receptor for nociceptin/orphanin FQ peptide (NOP) and μ-opioid peptide (MOP) receptor; and (b) dosing a patient once a day with an immediate release composition comprising cebranopadol or a pharmaceutically acceptable salt thereof which comprises a dual receptor for nociceptin/orphanin FQ peptide (NOP) and μ-opioid peptide (MOP) receptor. In certain embodiments, the regimen (a) comprises titrating down the dosage of a Class II, Class III or Class IV opioid or opioid agonist by decreasing the dose of the opioid or opioid agonist in (a) over the period of 1 to 3 days. In certain embodiments, steps (a) and (b) are performed during the same or overlapping time periods. In certain embodiments, opioid or mu opioid agonist is selected from tramadol, oxycodone, morphine, hydrocodone, fentanyl, oxymorphone, hydromorphone, buprenorphine, codeine, tapentadol, methadone, meperidine, or levorphanol. In certain embodiments, the mu agonist activity (or abuse potential) is assessed using a visual analog scale (VAS) rating, pupillometry and/or a Multi-Task Test. In certain embodiments, composition comprising the cebranopadol is a tablet. In certain embodiments, the tablet is film coated tablet. In certain embodiments, the side effects comprise nausea, vomiting, dizziness, pruritis, hyperhidrosis and/or hot flush sensation. In certain embodiments, the patient is renally or hepatically impaired. In certain embodiments, the cebranopadol is in free base form. In certain embodiments, at least 80% of the cebranopadol is crystal form A.

In certain embodiments, a method for treating pain with reduced risk of abuse is provided in a patient having nociceptive pain, said regimen comprising dosing a patient once daily with an immediate release composition comprising cebranopadol or a pharmaceutically acceptable salt thereof. In certain embodiments, the composition is a film-coated tablet. In certain embodiments, the cebranopadol is in free base form. In certain embodiments, at least 80% of the cebranopadol in the composition is in crystal form A.

In certain embodiments, a cebranopadol composition useful for reducing pruritus, hyperhidrosis, feeling hot and/or hot in a subject receiving analgesic treatment for pain is provided, which provides the analgesic therapeutic effect of an opioid. In certain embodiments, the cebranopadol is in free base form. In certain embodiments, at least 80% of the cebranopadol in the composition is in crystal form A.

In certain embodiments, a method for reducing pruritus, hyperhidrosis, feeling hot and/or hot in a subject receiving analgesic treatment for pain is provided, which provides the therapeutic effect of an opioid, comprising administering a composition comprising cebranopadol.

In one or more of these embodiments, uses and/or compositions, cebranopadol may be the sole active pharmaceutical ingredient in the composition and/or regimen.

It will be understood from the specification that the following examples are not limitations on the various embodiments of the invention.

### EXAMPLES

The cebranopadol oral human abuse potential study in the following example was a phase I single-dose, randomized, double-blind, five-way crossover study of 47 participants to evaluate the abuse potential of two supratherapeutic doses of cebranopadol in adult nondependent recreational opioid users versus placebo and commonly used opioids, oxycodone, a schedule II narcotic, and tramadol, a schedule IV narcotic. Eligible participants randomly received a single dose of placebo, cebranopadol 600 ug, cebranopadol 1000 ug, tramadol IR 600 mg, or oxycodone IR 40 mg. Abuse potential was determined based on participant-reported likeability using a Visual Analog Scale (VAS) following administration of study drug or placebo. Topline results show that one 600 μg dose of cebranopadol was similarly liked as placebo. Both 600 μg and 1000 μg doses of cebranopadol were liked significantly less than tramadol 600 mg (17.34, p<0.0001 and 7.77, p=0.0077, respectively) and oxycodone 40 mg (24.43, p<0.0001 and 14.86, p<0.0001, respectively), suggesting cebranopadol has significantly less potential for abuse compared to both schedule II and schedule IV narcotic.

Overall, both supratherapeutic dosages of cebranopadol did not raise any safety concerns. The most common reported adverse event was nausea, which was greatest after the administration of tramadol 600 mg (49%) versus either dose of cebranopadol 1000 μg and 600 μg (35% and 15%) or oxycodone (32%). The rate of vomiting after the administration of tramadol 600 mg and 1000 μg of cebranopadol was similar, 31% versus 30%. The fewest reports of vomiting were received when participants received 600 μg of cebranopadol (16%). Adverse events involving the nervous system were observed most often after the administration of tramadol with fewest reported after the administration of cebranopadol or placebo. Reports of pruritis (itchy skin) were greater after administration of oxycodone (30%) and tramadol (18%) when compared to 600 μg and 1000 μg of cebranopadol (4% and 7%).

### A. Illustrative Synthesis of Crystalline Form A

The following abbreviations are used in the examples: iBuOAc iso-butyl acetate; 1BuOH n-butanol (1-butanol); DMSO dimethyl sulfoxide; EtOAc ethyl acetate; EtOH ethanol; Ex example; FT-Raman Fourier transformation Raman spectroscopy; IPE diisopropyl ether; Δm change in mass; MeCN acetonitrile; MEK 2-butanone; MeOH methanol; min minute; NMP N-methyl-2-pyrrolidone; 1PrOH n-propanol (1-propanol); 2PrOH iso-propanol (2-propanol); PXRD powder x-ray diffraction; r.h. relative humidity; RT room temperature, preferably 20-25° C.; SCXRD single crystal X-ray diffraction; see seconds; t time (duration); TBME tert-butyl methyl ether; TG-FTIR thermogravimetry coupled with Fourier transform infrared spectroscopy; THF tetrahydrofuran; XRPD X-ray powder diffraction. Unless otherwise specified, solvent mixtures are always volume/volume.

100 mg (1r,4r)-6′-fluoro-N,N-dimethyl-4-phenyl-4′,9′-dihydro-3′H-spiro[cyclohexane-1,1′-pyrano[3,4,b]indol]-4-amine [crystalline form D according to D)] was suspended in 0.5 mL TBME. The suspension was stirred at RT for six days. The resulting solid was filtered out and dried in air. A crystalline solid of crystalline form A was obtained and characterized by PXRD.

The following Table shows the peak list for crystalline form A. The uncertainty in the 2θ values is ±0.2° in 2θ; rel. I is the relative intensity of the respective peaks. Maximum intensity is 100

| 2 θ | d value Å | Intensity Cps | rel. I % |
|---|---|---|---|
| 7.8 | 11.3 | 324 | 16 |
| 8.8 | 10.1 | 156 | 8 |
| 9.1 | 9.7 | 120 | 6 |
| 10.5 | 8.4 | 40 | 2 |
| 11.1 | 8.0 | 300 | 15 |
| 11.7 | 7.6 | 64 | 3 |
| 12.2 | 7.3 | 280 | 14 |
| 13.5 | 6.5 | 343 | 17 |
| 15.2 | 5.8 | 140 | 7 |
| 16.0 | 5.5 | 134 | 7 |
| 16.8 | 5.3 | 347 | 17 |
| 17.6 | 5.1 | 735 | 36 |
| 18.3 | 4.9 | 2044 | 100 |
| 18.6 | 4.8 | 1295 | 63 |
| 19.4 | 4.6 | 474 | 23 |
| 19.7 | 4.5 | 346 | 17 |
| 20.6 | 4.3 | 214 | 11 |
| 20.8 | 4.3 | 308 | 15 |
| 21.5 | 4.1 | 178 | 9 |
| 22.0 | 4.0 | 135 | 7 |
| 22.3 | 4.0 | 199 | 10 |
| 22.6 | 3.9 | 227 | 11 |
| 23.5 | 3.8 | 263 | 13 |
| 23.9 | 3.7 | 208 | 10 |
| 24.2 | 3.7 | 176 | 9 |
| 25.0 | 3.6 | 292 | 14 |
| 25.8 | 3.5 | 505 | 25 |
| 26.3 | 3.4 | 564 | 28 |
| 27.2 | 3.3 | 180 | 9 |
| 27.6 | 3.2 | 442 | 22 |
| 28.3 | 3.2 | 360 | 18 |
| 29.1 | 3.1 | 249 | 12 |
| 29.5 | 3.0 | 115 | 6 |
| 30.0 | 3.0 | 256 | 13 |
| 31.6 | 2.8 | 147 | 7 |
| 32.3 | 2.8 | 174 | 9 |
| 32.6 | 2.8 | 147 | 7 |
| 33.8 | 2.7 | 96 | 5 |

### Analysis—DVS

Crystalline Form A was previously characterized by dynamic vapor sorption (DVS) using a Projekt Messtechnik SPS 11-100n multi sample water vapor sorption analyzer. For the DVS analysis, each sample was allowed to equilibrate at 50% r.h. (relative humidity) before starting a pre-defined humidity program during which the change in weight of the sample is determined. All measurements were performed according to the following program: 2 h at 50% r.h.; 50% r. h.→0% r.h. (10%/h); 5 h at 0% r.h.; 0→95% r.h. (5%/h); 3 h at 95% r.h.; 95→50% (10%/h), and 2 h at 50% r.h.

Although hygroscopicity was measured in a slightly different manner, it was classified according to the European Pharmacopoeia as follows: very hygroscopic (vh): increase of the mass≥15%; hygroscopic (h): increase of the mass is less than 15% and equal or greater than 2%; slightly hygroscopic (sh): increase of the mass is less than 2% and equal or greater than 0.2%; not hygroscopic (nh): increase of the mass is less than 0.2%; deliquescent (d): sufficient water is absorbed to form a liquid.

DVS with two cycles was performed on a sample of crystalline form A. The first cycle was not symmetric, the sample contained still water when the DVS cycle returned to 50% r.h. (relative humidity in %). The second cycle was reversible. Below 40% r.h. the relative mass returned to ~100% (water content=0%). The hysteresis between 40% and 70% r.h. indicates a metastable zone. The second cycle indicated the following transformations: hemi-hydrate→ansolvate (<38% r. h.)→hemi-hydrate (>70% r. h.). The sample was classified to be hygroscopic (Δm=3-4% at 85% r. h.; Δm: change in mass)

Example 1: A single-dose, randomized, double-blind, placebo- and active-controlled crossover trial to evaluate the abuse potential of two doses of cebranopadol in adult nondependent recreational opioid users.

Investigational Medicinal Products (IMPs): Study Design: Randomized, single site, double-blind, placebo- and active-controlled, crossover, single oral dose, Phase 1 trial, in non-dependent recreational opioid users

Cebranopadol film coated tablets, Oxycodone HCl tablets, Tramadol HCL tablets, and matching placebos.

### Objectives:

Primary Objective: To evaluate the abuse potential of single doses of cebranopadol relative to oxycodone immediate-release (IR), tramadol IR, and placebo in non-dependent recreational opioid users

### Secondary Objectives:

To evaluate the abuse potential of oxycodone IR and tramadol IR compared with placebo.
To evaluate the safety and tolerability of cebranopadol
To evaluate the pharmacokinetics (PK) of cebranopadol and active metabolites M2, M3, and M6

### Study Treatments:

Qualification Phase: Based on the assigned treatment sequence, each subject will be randomly allocated to receive a single oral dose of the IMP on each day of the Qualification Phase. On each day, subjects will receive after an overnight fast, a single oral dose of 6 identically appearing capsules that will contain one of the following treatments:
Oxycodone HCl tablets, 20 mg: single oral dose of 40 mg.
Tramadol HCL tablets, 100 mg: single oral dose of 600 mg.
Matching placebo

Treatment Phase: Based on the assigned treatment sequence, each subject will be randomly allocated to receive a single oral dose of the IMP in each of the 5 periods. At each period, subjects will receive an oral single dose of 6 identically appearing capsules that will contain one of the following treatments:
Cebranopadol film coated tablets, 200 μg: single oral dose of 600 μg.
Cebranopadol film coated tablets, 200 μg: single oral dose of 1000 μg.

| Ingredients for 200 μg | Amount per | |
|---|---|---|
| film-coated tablet | tablet [mg] | Function |
| Cebranopadol, micronized | 0.200 | API |
| Lactose monohydrate | 50.05 | filler |
| Microcrystalline cellulose | 40.00 | filler |
| Croscarmellose sodium | 3.00 | disintegrant |
| Povidone K30 | 5.00 | binder |
| Sodium lauryl sulphate | 1.00 | surfactant |
| Magnesium stearate | 0.75 | lubricant |
| Opadry ® AMB II white | 4.00 | film-coat |
| Total weight | 104.00 | — |

Oxycodone HCl tablets, 20 mg: single oral dose of 40 mg (Positive control; Schedule II opioid). The 40 mg dose is expected to show significant abuse-related subjective effects without interfering with completion of PD measures or producing aversive effects, and although higher doses have been shown to produce higher positive effects without significant adverse effects, 40 mg is expected to be sufficient to demonstrate a difference in all endpoints between oxycodone IR and placebo.
Tramadol HCL tablets, 100 mg: single oral dose 600 mg (Positive control; Schedule IV opioid). In human laboratory studies, tramadol IR appears to have a lower abuse potential compared with other opioids (most commonly Schedule II: see Dunn et al., 2019, for systematic review7); however, the effects of tramadol IR in a human abuse potential study can vary based on route of administration and if the subject sample is physically dependent on opioids. Oral tramadol IR doses ranging between 25 mg to 700 mg have been evaluated in non-physically dependent individuals. The dose of tramadol IR in the present study (600 mg) has been selected based on variable findings of liking at doses≤400 mg and because the 700 mg dose was shown to be safe and reasonably well tolerated in a similar subject population. [Babalonis S, Lofwall M R, Nuzzo P A, Siegel A J, Walsh S L. Abuse liability and reinforcing efficacy of oral tramadol in humans. Drug Alcohol Depend. 2013 Apr. 1; 129 (1-2): 116-24; Duke A N, Bigelow G E, Lanier R K, Strain E C. Discriminative stimulus effects of tramadol in humans. J Pharmacol Exp Ther. 2011 July; 338 (1): 255-62. Doi: 10.1124/jpet. 111.181131. Epub 2011 Apr. 5. PMID: 21467190; PMCID: PMC3126638; Epstein D H, Preston K L, Jasinski D R. Abuse liability, behavioral pharmacology, and physical-dependence potential of opioids in humans and laboratory animals: lessons from tramadol. Biol Psychol. 2006 July; 73 (1): 90-9. Doi: 10.1016/j.biopsycho.2006.01.010. Epub 2006 Feb. 23. PMID: 16497429; PMCID: PMC2943845]. However, in those studies, subjects did not undergo a Qualification Phase to confirm that they could discriminate and like the effects of tramadol IR relative to placebo in contrast to the present trial.

### Matching Placebo(s)

| | | |
|---|---|---|
| | # Capsules containing | # Placebo |
| Assigned Treatment | Active Treatment | Capsules |

Treatment Phase

| | | |
|---|---|---|
| Oxycodone HCl 40 mg (20 mg | 2 | 4 |
| tablets) | | |
| Tramadol HCl 600 mg (100 mg | 6 | 0 |
| tablets) | | |
| Cebranopadol 600 μg (200 μg | 3 | 3 |
| film coated tablets) | | |
| Cebranopadol 1000 μg (200 μg | 5 | 1 |
| film coated tablets) | | |
| Placebo | 0 | 6 |

*Naloxone will be used for the naloxone challenge test on Day-1 of the Qualification Phase to confirm that subjects are not physically dependent on opioids. The subcutaneous dose in the upper arm will be 0.8 mg (2.0 mL) of naloxone.

Study Centers: Single center in the US
Study Duration: Individual subject participation is expected to be up to ~4 months, including an Enrollment period up to 4 weeks.
Sample Size: 50 adult men and women total with the intention of at least 38 completing the study and being included in the primary analysis population (Modified Completer Population).
Study Population: The study will enroll healthy men and women, 18 to 55 years old, with a history of recreational opioid use defined as nontherapeutic use of opioids at least 10 times in the subject's lifetime and at least once in the 12 weeks prior to the Enrollment Visit.

### Inclusion Criteria for Enrollment:

1. Willing and able to provide written informed consent
2. Adult men or women aged 18 to 55 years, inclusive
3. History of recreational opioid use defined as non-therapeutic use at least 10 times in the subject's lifetime and at least once in the 12 weeks prior to the Enrollment Visit
4 Body mass index between 19 kg/m² and 32 kg/m² inclusive, with a body weight of not less than 50 kg at Enrollment
5. Subjects must be in good health as determined by medical history, physical examination, 12-lead electrocardiogram (ECG), and vital signs (pulse rate, systolic blood pressure and diastolic blood pressure, respiratory rate, and oxygen saturation using pulse oximetry) at Enrollment
6. Females who participate in this study will be of childbearing or non-childbearing potential.
   Childbearing potential: Physically capable of becoming pregnant.
   Non-childbearing potential:
   Permanently sterile (i.e., both ovaries removed, uterus removed, or bilateral tubal ligation for at least 6 weeks or documented successful hysteroscopic sterilization); and/or
   Post-menopausal (no menstrual period for at least 12 consecutive months without any other medical cause)
7. Females of childbearing potential must be non-lactating and must have a negative serum pregnancy test at Screening
8. Willing to use acceptable, effective methods of contraception
9 Male subjects must agree not to donate sperm for at least 4 weeks after the final visit
10. Be able to attend the clinic regularly and reliably
11. Be able to understand, read, write, and speak English fluently to complete the study related materials
12. Be informed of the nature of the study and give written consent prior to any study procedure

### Exclusion Criteria for Enrollment:

1 Self-reported history of drug or alcohol dependence (lifetime) other than caffeine or nicotine as defined by DSM-IV-TR criteria
2. Current treatment or treatment within their lifetime for substance disorders, other than treatment for smoking cessation
3. Positive or missing alcohol breath test at Enrollment; the alcohol breath test can be repeated and/or the subject rescheduled at the discretion of the investigator or designee
4. Pregnant or breastfeeding or missing pregnancy test
5. Unwillingness or inability to abstain from recreational drug use for the duration of the trial
6. Current consumption of greater than 20 cigarettes per day or inability to abstain from smoking (or use of any nicotine-containing substance) for at least 8 hours
7. Participation in another clinical trial within 30 days prior to Enrollment that resulted in the administration of at least 1 dose of IMP
8 Diseases or conditions known to interfere with the absorption, distribution, metabolism, or excretion of drugs. Subjects with a history of cholecystectomy are not excluded
9 Prolongation of QTcF (after repeated assessment) at Enrollment, i.e., >450 ms for men or >470 ms for women, or presence of additional risk factors for torsade de pointes (e.g., heart failure, hypokalemia), or use of concomitant medications that prolong the QT interval
10. History of orthostatic hypotension or other cardiovascular diseases
11. Any clinically significant disease that in the investigator's opinion may affect efficacy or safety assessments or may compromise the subject's safety during trial participation, e.g., significant pulmonary, gastrointestinal, cardiac, endocrine, metabolic, neurological, or psychiatric disorders
12. Definite or suspected history of drug allergy or hypersensitivity to opioids or opioid antagonists
13. Use of prescription medications within the longer of 14 days or 5 half-lives or use of over-the-counter medications within the longer of 7 days or 5 half-lives prior to dosing, exceptions may be made on a case-by-case basis (e.g., for medications with a short half-life or for topical medications) if approved by the medical monitor in agreement with the investigator
14. Any contraindication for naloxone, oxycodone IR, or tramadol IR administration
15. Not able to abstain from consumption of:
   Beverages or food containing caffeine (tea, coffee, cola, chocolate, etc.) or alcohol from 2 days prior to each Day 1 until discharge from the research unit.
   Beverages or food containing quinine (e.g., bitter lemon, tonic water) from 1 week before Day 1 of the Qualification Phase until the final examination.
   Grapefruit juice (sweet or sour) or Seville oranges from 1 week before Day 1 of the Qualification Phase until the final examination.
16. Blood loss of 500 mL or more within 4 weeks before dosing in the treatment phase in this trial, including blood donation. Planned blood donations during the trial and up to 12 weeks after the Final Examination
17. History of seizure disorder including unprovoked seizure and/or epilepsy or any condition associated with a significant risk for seizure disorder or epilepsy at the Enrollment Visit at the discretion of the investigator
18. Known or suspected of not being able to comply with the trial protocol
19. Not able to communicate meaningfully with the investigator or trial site staff
20. Employee of the investigator or trial site, with direct involvement in the proposed trial or other trials under the direction of that investigator or trial site, as well as family members of the employees or the investigator
21. Considered by the Investigator not to be suitable for the study for any other reason

### Exclusion Criteria for Day 1 (Qualification Phase):

1. Positive naloxone challenge on Day-1 of the Qualification Phase
2. Use of forbidden medication since the Enrollment Visit (within 7 days for OTC, 14 days for prescription)
3. Positive or missing pregnancy test
4. Positive or missing alcohol breath test; alcohol breath test may be repeated or rescheduled at the discretion of the investigator or designee
5. Positive or missing urine drug of abuse screen result, except for cannabinoids (tetrahydrocannabinol [THC])
6. Positive or missing viral serology, i.e., human immunodeficiency virus Type 1 and Type 2 antibodies and antigen, hepatitis B surface antigens, anti-HBC and IgM anti-HBc, and hepatitis C virus antibodies, based on sample taken at the Enrollment Visit
7. Any abnormal laboratory values or any clinically relevant out-of-range values for safety laboratory parameters (clinical chemistry, coagulation, hematology, and urinalysis) based on sample taken at the Enrollment Visit, as judged by the investigator
8 Blood donation or acute loss of blood (more than 100 mL) since the Enrollment excluding blood samples required by the protocol
9. Any relevant deterioration in the health of the subject that could alter the benefit/risk assessment for the subject, including adverse events (AEs), laboratory parameters, vital signs, or other safety parameters (e.g., ECGs)
10. Failure to comply with trial requirements, e.g., intake of forbidden medications, consumption of alcohol, considered by the investigator to affect subject safety or interfere with the integrity of the trial
11. Uncooperative subjects or subjects who refused to continue in the trial
12. Withdrawal of informed consent
13. Any contraindication for naloxone, oxycodone IR, or tramadol IR administration.

### Discontinuation Criteria Following Qualification and Prior to Treatment Phase:

1. Failure to successfully fulfill any of the following criteria based on the qualification phase prior to the first treatment period (Treatment Period 1, Day-1):
   Peak score in response to oxycodone IR and tramadol IR is numerically greater than that of placebo on “at the moment” Drug Liking VAS (difference of at least 15 points), with a minimum score of 65 points.
   Acceptable placebo response based on Drug Liking VAS, defined as a peak score between 40 and 60 points (a score that is considered neutral on the scale as neither like nor dislike), inclusive
   Acceptable overall responses to oxycodone IR 40 mg, tramadol IR 600 mg, and placebo on the subjective measures, as judged by the investigator or designee and sponsor
   The ability to tolerate oxycodone IR and tramadol IR, as judged by the investigator based on available safety data (e.g., respiratory rate≥8 breaths/min and no vomiting within 4 hours after dosing).
   General behavior suggestive that they could successfully complete the trial, as judged by the investigator
2. Positive or missing alcohol breath test; alcohol breath test may be repeated or rescheduled at the discretion of the investigator or designee
3. If a subject tests positive for other drugs of abuse, except for cannabinoids (THC), the urine drug screen can be repeated and/or the subject rescheduled at the discretion of the investigator or designee

Methodology: The study comprises the Enrollment Visit, a Qualification Phase, a Treatment Phase consisting of 5 treatment periods, and an End of Study Visit. See Section 1.2 for a tabular schedule of events for the Qualification Phase and Section 1.3 for the Treatment Phase (which lists all assessments planned).

### Enrollment Visit

Enrollment will begin no more than 28 days prior to the first dose of study medication. After completing the informed consent process and signing the informed consent form, subjects will undergo study-specific Enrollment procedures. A physical examination, including measurement of height, weight, and vital signs; blood and urine laboratory testing; review of medical and medication history; a 12-lead ECG will be performed, as well as the Columbia-Suicide Severity Rating Scale (C-SSRS) and an assessment of current substance dependence (DSM-IV-TR).

### Naloxone Challenge:

Subjects will check-in on Day-1 of the Qualification Phase following the return of a negative urine drug screen test, excluding THC. Subjects with a positive urine drug screen test for opioids will be screen failed and dismissed from the research clinic. Subjects who remain in the clinic on Day-1 will undergo a naloxone challenge test to exclude the possibility of physical dependence on opioids. The Objective Opioid Withdrawal Scale (OOWS) will be used to record any signs or symptoms of withdrawal observed during the naloxone challenge test.

### Qualification Phase

Each subject will attend a double-blind Qualification Phase consisting of a 4-night confinement period during which they will receive, after an overnight fast, a single oral dose of 6 identically appearing capsules that will contain one of the following: oxycodone IR 40 mg, tramadol IR 600 mg, or placebo in a randomized crossover manner separated by ~24 hours. Pharmacodynamic and safety assessments will be performed from pre-dose through 8 hours after each IMP administration. The purpose of the Qualification Phase is to ensure that the subjects can discriminate between active drug and placebo, can tolerate oxycodone IR 40 mg and tramadol IR 600 mg, can feel comfortable with the pharmacodynamic measures, can follow directions, and are cooperative.

Subjects will be confined to the trial site from Day-1 until 72 hours after the first IMP administration in the Qualification Phase.

### Treatment Phase

There will be a washout period of at least 72 hours between the end of the Qualification Phase and the beginning of the first treatment period. Based on the assigned treatment sequence, each subject will be randomly allocated to receive a single oral dose of 6 identically appearing capsules in each of the 5 periods. Each IMP administration will be given under fasted conditions and will be separated by a washout period of at least 14 days between treatments.

Subjects will be confined to the trial site from Day-1 until 48 hours after IMP administration in each of the treatment periods. Pharmacodynamic, safety and pharmacokinetic (PK; blood sampling) assessments will be performed from pre-dose through 48 hours post-dose in each treatment period.

An End of Study Visit will be conducted at 5-10 days after discharge from the last treatment period or upon early discontinuation from the trial.

### Concomitant Medications

No concomitant medication (including prescription drugs, over-the-counter drugs, and herbal remedies like St. John's wort) will be allowed during the trial, with the exception of acetaminophen (e.g., for headache), and the continuous use of hormonal contraceptives.

### Data Collection and Assessment:

Demographics and other subject characteristics: Demographic data will comprise sex, age, height, weight, race/ethnicity, and the use of nicotine products. Body mass index will be calculated. Other subject characteristics will comprise medical history (medical history and surgical interventions), recreational drug and alcohol use history, prior and concomitant medications.

### Pharmacodynamics:

### Primary Measure:

Visual analog scale (VAS) rating for Drug Liking “at this moment” Key Secondary Measures:
   VAS rating for Overall Drug Liking
   VAS rating for Take Drug Again

### Other Secondary Measures:

VAS rating for Any Drug Effects, High, Good Drug Effects, Bad Drug

### Effects, Feeling Sick, Alertness/Drowsiness, Floating, and Detached.

VAS rating for Drug Similarity.
Pupillometry.
Multi-Task Test

### Pharmacokinetics

Plasma concentrations of cebranopadol and active metabolites M2, M3, and M6, oxycodone IR and tramadol IR will be assayed. Descriptive PK parameters will be derived from plasma concentration-time data using noncompartmental methods: Cₘₐₓ, Tₘₐₓ, AUC₀₋ₜ, and AUC_{inf}. A separate PK report will be generated.

Pharmacokinetic Parameters to be Calculated from Plasma Concentration-Time Data

| Parameter | Description |
|---|---|
| AUC₀₋ₜ | Area under the concentration-time curve up to |
| | the sampling time t. If it is not replaced by a |
| | numerical value, then t is the last sampling |
| | time with quantifiable |
| | concentration and this parameter will not |
| | contain an extrapolated portion. |
| AUC_{inf} | Area under the concentration-time curve from |
| | time 0 to infinite time. |
| Cmax | Maximum observed concentration. |
| Tmax | Time to attain maximum concentration. |

Concentrations below the lower limit of quantification (except for data points before the first quantifiable concentration) will be excluded from the PK analysis and descriptive statistics will only be determined if at least 75% of the observations at each sampling point have quantifiable results. If a pre-dose concentration is above the assay quantification limit and lower than 5% of Cₘₐₓ, it will be set to zero in the PK analysis. Subjects with higher pre-dose values will be excluded from the PK analysis.

Areas under the curves will be calculated using the log-linear trapezoidal rule, i.e., linear up to the maximum concentration and log thereafter.

The individual concentrations will be listed for each measurement time point and for each period and analyzed descriptively. Tabulated values will be rounded to 3 significant figures. Statistical calculations will be performed with values that have not been rounded.

Mean, individual, and overlay concentration-time profiles will be plotted on both linear and semi-logarithmic scales on the same, portrait-oriented page.

### Safety

Safety data will comprise physical examinations, oral body temperature, vital signs (pulse rate, systolic blood pressure, diastolic blood pressure, respiratory rate, and oxygen saturation using pulse oximetry), 12-lead ECG, adverse events (AEs), telemetric safety monitoring (5-lead ECG, oxygen saturation, and pulse rate), C-SSRS, safety laboratory parameters (clinical chemistry, coagulation, hematology, and urinalysis), and pregnancy test (females of childbearing potential only).

### Statistical Methods:

### Analysis Populations

Qualification Safety Population: All subjects who receive at least one dose of study drug in the Qualification Phase. All safety evaluations in the Qualification Phase will be performed using this population.

Randomized Population: All subjects who are assigned a randomization number in the Treatment Phase.

Safety Population: All subjects who receive at least one dose of study drug in the Treatment Phase. All safety evaluations in the Treatment Phase will be performed using this Safety Population.

Completer Population: All randomized subjects who complete all treatment periods of the Treatment Phase and have at least one response on the VAS for Drug Liking within 2 hours of Tₘₐₓ for each treatment or, in the case of placebo, at least one response on the VAS for Drug Liking. If Tₘₐₓ is missing or inestimable for a given subject/treatment (other than placebo), the median Tₘₐₓ for that treatment for subjects in the Completer Population with non-missing Tₘₐₓ will be used for this determination.

Modified Completer Population: All subjects in the Completer Population, excluding subjects with similar Emax scores (within 5 points difference) across all study treatments (including placebo) or subjects with an Emax for placebo >60 AND the difference between Emax for placebo and oxycodone IR is ≤5. This population will serve as the primary population for the PD analyses.

Pharmacokinetic Population: All subjects who receive at least one dose of IMP and have at least one measurable PK sample after dosing. All PK evaluations in the Treatment Phase will be performed using the PK Population.

### Pharmacodynamic Analyses

All analyses will be performed using the Modified Completer Population. If the Modified Completer Population and Completer Population differ by more than 10%, Drug Liking Visual Analog Scale (VAS) is analyzed and reported for the Completer Population. The following summary parameters will be calculated for all assessments except for pupillometry and Overall Drug Liking, Take Drug Again, and Drug Similarity VAS:
Peak effect (Eₘₐₓ and/or Eₘᵢₙ)
Time of peak effect (T_{Emax} and/or T_{Emin}).
Area under the effect curve to 1 hour (AUE₀₋₁ₕ).
Area under the effect curve to 8 hours (AUE₀₋₈ₕ)

For Overall Drug Liking and Take Drug Again VAS, Eₘₐₓ and Eₘᵢₙ will be calculated. For Drug Similarity VAS, a descriptive analysis will be conducted. For pupillometry, the following summary parameters will be calculated:
Maximum pupil constriction (MPC)
Time of MPC (TMPC)
Pupillometry area over the effect curve to 1 hour (PAOC₀₋₁ₕ).
Pupillometry area over the effect curve to 8 hours (PAOC₀₋₈ₕ)
   The primary endpoint will be Drug Liking VAS Eₘₐₓ and the key secondary endpoints will be Overall Drug Liking VAS Eₘₐₓ and Take Drug Again VAS Eₘₐₓ.

### Primary Endpoint Analysis: Drug Liking VAS Eₘₐₓ

The primary PD endpoint (Drug Liking VAS Eₘₐₓ) will be evaluated with a linear mixed effects model containing treatment, period, sequence, and first-order carryover as fixed effects and subject nested within sequence as random effect, using the Modified Completer Population. A supportive analysis of the primary endpoint may be performed using the Completer Population if this population differs from that of the Completer Population by more than 10%. This supportive analysis will utilize the same hypotheses as that of the primary analysis. The first order carryover effect will be the previous treatment received in the Treatment Phase. If the carryover effect is found to be non-significant at the 25% level, then the term will be dropped from the model. If the carryover effect is significant at the 25% level, but not at the 5% level, then the carryover effect term will be retained in the model; if the carryover effect is significant at the 5% level, a first period analysis will be conducted.

From the model, least squares means, and confidence intervals (CIs) will be provided for each treatment, and difference in least squares mean, CIs of the difference, and p-values will be provided for each treatment comparison. Unless otherwise indicated, CIs will be 2-sided at the 90% level, p-values will be 1-sided, and significance testing will be performed at 1-sided significance level 0.05.

Levene's test will be used to evaluate potential heterogeneity of variance in the model with a one-way analysis of variance (ANOVA), including residuals as the response and treatment as a fixed effect. If the p-value is not significant at the 0.05 level, the mixed model with equal variances will be performed. If the p-value of the Levene's test is ≤0.05, it will be concluded that there is a difference in variance among treatments, and the model will be corrected by estimating the variances for treatment separately (unequal variance model using the Satterthwaite method and repeated statement).

The residuals from the mixed-effects model will be investigated for normality using the Shapiro-Wilk W test. Parameters will be analyzed under the assumption of a normal distribution of errors if the p-value of the test is >0.01, and the mixed effects model will be used for reporting for the final analysis.

If the p-value is <0.01 for the Shapiro Wilk W test on the residuals from the mixed model, a test of skewness will be conducted on each paired difference. If the distribution of the paired differences is not skewed (−0.5<skewness value<0.5), then Drug Liking Eₘₐₓ will be analyzed using paired t-tests for each treatment comparison. If the distribution of the paired differences is skewed (skewness value<−0.5 or skewness value>0.5), then Drug Liking Eₘₐₓ will be analyzed non-parametrically. The Sign Test will be used to evaluate treatment differences. If a paired t-test is chosen for Drug Liking Eₘₐₓ, means, mean differences and corresponding one-sided 95% CIs, as well as p-values for the appropriate hypothesis will be presented. If a Sign Test is chosen, medians from each treatment as well as medians, and first and third quartiles of the differences between treatments, and corresponding Sign Test p-values will be provided for each treatment comparison. The overall treatment effect will be assessed using Friedman's test.

If a first period analysis is conducted, Drug Liking VAS Eₘₐₓ will be analyzed using an ANOVA model containing treatment group as the fixed effect. Least squares means and 95% CIs will be provided for each treatment, and difference in least squares mean, one-sided 95% CIs of the difference, and p-values will be provided for each treatment comparison subject to a hypothesis test.

The comparisons of interest are as follows:
Cebranopadol vs. placebo.
Cebranopadol vs. oxycodone IR.
Cebranopadol vs. tramadol IR
Oxycodone IR vs. placebo (study validity)
Tramadol IR vs. placebo

For study validity, the primary endpoint, Drug Liking Eₘₐₓ, will be compared between oxycodone IR (primary positive control) and placebo, the following hypothesis will be tested:

$$Ho : μ C 1 - μ P \leq 15 vs . Ha : μ C 1 - μ P > 15 \qquad ( 1 )$$

where μC1 is the mean for oxycodone IR and μP is mean for placebo. The margin of 15 was selected based on previous studies of this type. Pathak S, Vince B, Kelsh D, et al. Abuse potential of samidorphan: A Phase I, Oxycodone-, pentazocine-, naltrexone-, and placebo-controlled study. J Clin Pharmacol. 2019 February; 59 (2): 218-228; Setnik B, Roland C L, Cleveland J M, Webster L. The abuse potential of Remoxy®, an extended-release formulation of Oxycodone, compared with immediate- and extended release Oxycodone. Pain Med. 2011 April; 12 (4): 618-31]. If the treatment difference of oxycodone IR compared with placebo is statistically significant at alpha level 0.05, validity is established for the study.

The evaluation of abuse potential of tramadol IR (secondary positive control) will be the comparison of tramadol IR versus placebo. Despite an absence of published literature on liking of tramadol IR (using a bipolar VAS among qualified recreational drug users), a margin of 15 has been selected. The following hypothesis will be tested for tramadol IR compared with placebo:

$$Ho : μ C 2 - μ P \leq 15 vs . Ha : μ C 2 - μ P > 15 \qquad ( 2 )$$

where μC2 is the mean for tramadol IR and μP is mean for placebo. If tramadol IR fails the validation test, results of all pairwise comparisons with tramadol IR from the model will be considered descriptive. Failure of tramadol IR to separate from placebo will not impact subsequent tests between oxycodone IR, cebranopadol and placebo.

The primary treatment comparison for relative abuse potential of cebranopadol will be the comparison of Drug Liking Eₘₐₓ between cebranopadol at each dose level and oxycodone IR. The following hypothesis will be tested for each comparison of cebranopadol and oxycodone IR:
Cebranopadol 600 μg vs. oxycodone IR 40 mg
Cebranopadol 1000 μg vs. oxycodone IR 40 mg

$$Ho : μ C 1 - μ T \leq 0 vs . Ha : μ C 1 - μ T > 0 \qquad ( 3 )$$

where μC1 is the mean for oxycodone IR and μT is mean for cebranopadol.

The secondary treatment comparisons for relative abuse potential of cebranopadol will be the comparison of cebranopadol at each dose level versus tramadol IR. For each comparison, the following hypothesis will be tested:
Cebranopadol 600 μg vs. tramadol IR 600 mg.
Cebranopadol 1000 μg vs. tramadol IR 600 mg

$$Ho : μ C 2 - μ T \leq 0 vs . Ha : μ C 2 - μ T > 0 \qquad ( 4 )$$

where μC2 is the mean for tramadol IR and μT is mean for cebranopadol.

The evaluation of absolute abuse potential of cebranopadol will be the comparison of cebranopadol versus placebo. The following hypothesis will be tested:
Cebranopadol 600 μg vs. placebo.
Cebranopadol 1000 μg vs. placebo

$$Ho : μ T - μ P \leq 11 vs . Ha : μ T - μ P < 11 \qquad ( 5 )$$

where μT is the mean for cebranopadol and μP is mean for placebo.

A significance level of 0.05 will be used for all 1-sided tests. As the hypotheses will be tested sequentially and must be met at all dose levels, no adjustments in p-values will be made to account for multiple comparisons.

### Key Secondary and Other Secondary Endpoint Analysis:

All secondary PD endpoints, including the key secondary endpoints, are analyzed with the same approach as described above for the primary endpoint analysis, using the Modified Completer Population. From the model, least squares means, and CIs are provided for each treatment. Difference in least squares means and 2-sided 90% CIs for the difference is provided for each of the 5 treatment comparisons: oxycodone IR versus placebo, tramadol IR versus placebo, cebranopadol versus oxycodone IR, cebranopadol versus tramadol IR, and cebranopadol versus placebo.

Comparisons among treatments for secondary endpoints is evaluated at 1-sided significance level of 0.05 using the hypotheses shown below. No specific margins have been selected for secondary endpoints as there is currently no scientific literature to support selection of such margins.
1. Primary positive control (oxycodone)) (C1) vs. placebo (P):

$$H 0 : μ C 1 - μ P \leq 0 vs . Ha : μ C 1 - μ P > 0$$

2. Secondary positive control (tramadol IR) (C2) vs. placebo (P):

$$H 0 : μ C 2 - μ P \leq 0 vs . Ha : μ C 2 - μ P > 0$$

3. Primary positive control (oxycodone) (C1) vs. each dose of cebranopadol (T):

$$H 0 : μ C 1 - μ T \leq 0 vs . Ha : μ C 1 - μ T > 0$$

4. Secondary positive control (tramadol IR) (C2) vs. each dose of cebranopadol (T):

$$H 0 : μ C 2 - μ T \leq 0 vs . Ha : μ C 2 - μ T > 0$$

The direction of the hypotheses may be reversed for some endpoints (e.g., Eₘᵢₙ for Alertness/Drowsiness).

Comparisons for secondary endpoints between cebranopadol and placebo will be evaluated from 2-sided 90% CIs (α=0.10) using the confirmatory type of hypothesis as shown below:
5. Each dose of cebranopadol (T) vs. placebo (P):

$$H 0 : μ T - μ P = 0 vs . Ha : μ T - μ P \neq 0$$

No adjustments for p-values will be made to account for multiple comparisons in the analysis of secondary endpoints.

### Pharmacokinetic/Pharmacodynamic Modeling and Simulations

Methods and results of the pharmacokinetic/pharmacodynamic modeling and simulation analyses will be provided in separate report(s).

Safety analyses: All analyses will be performed using the Safety Population. All safety data will be analyzed as described in the section general descriptive and graphical methods. Potentially clinically significant values will be defined for various parameters, and the results will be summarized in tables. Additional details will be provided in the statistical analysis plan. All AEs will be listed together with information on onset, duration, frequency, intensity, seriousness, expectedness, relationship, outcome, and countermeasures taken.

This study used a randomized, double-blind, five-way crossover design to evaluate the abuse potential of cebranopadol in adult nondependent recreational opioid users versus placebo, oxycodone, and tramadol. Eligible subjects underwent a naloxone challenge to confirm nondependence to opioids, and a qualification phase to assess that subjects could tolerate oxycodone and tramadol and discriminate their effects from placebo. Qualified subjects underwent a >72-hour washout before receiving study drug in the Treatment Phase. Subjects were randomized to receive single doses of cebranopadol 600 ug or 1000 ug, oxycodone IR 40 mg, tramadol IR 600 mg, or placebo in a crossover manner. Each treatment period was separated by a ≥14-day washout period to prevent carryover effects. The primary endpoint was maximum drug liking “at this moment” measured using a bipolar 100-point Visual Analog Scale (VAS). Key secondary measures included “overall drug liking” and “take drug again” measured by VAS.

Results: Thirty-eight subjects completed the study, and 33 met criteria for inclusion in the Modified Completers population (pharmacodynamic analysis). For the primary endpoint of maximum drug liking “at this moment,” both cebranopadol 600 ug and 1000 ug were liked significantly less than tramadol 600 mg (17.34; 95% CI [10.99,23.70] and 7.77; [1.51,14.03], respectively) and oxycodone 40 mg (24.43; [18.34,30.52] and 14.86; [8.80,20.91], respectively). Results of the key secondary endpoints “overall drug liking” and “take drug again” were generally consistent with the primary endpoint. Neither dose of cebranopadol raised safety concerns, but two subjects experienced seizures after receiving tramadol. The most commonly reported adverse event was nausea.

Conclusions: This study demonstrated that cebranopadol has significantly lower abuse potential compared to both Schedule II (oxycodone) and Schedule IV (tramadol) opioids. This study confirms what has previously been established while furthering the understanding of the abuse potential of cebranopadol. Cebranopadol may serve as a much-needed alternative treatment option for patients with moderate to severe pain.

Unlike after treatment with oxycodone and tramadol, administration of cebranopadol did not produce pruritus, hyperhidrosis, feeling hot and/or hot flushing, that have been associated with the use of opioid analgesics. Specifically, when the incidence of subjects experiencing pruritis was analyzed, treatment with cebranopadol 600 μg and 1000 μg resulted in only 6.7% (n=3) and 7% (n=3) verses 29.5% (n=13) after having taken oxycodone 40 mg and 25.6% (n=10) after having taken tramadol 600 mg. A similar trend in frequency was observed for the AE hyperhidrosis; no subjects experienced the AE after taking either dose of cebranopadol verses 11.4% (n=5) after taking oxycodone and 12.8% (n=5) after tramadol. Moreover, a single subject reported feeling hot after receiving oxycodone (2.3%) and tramadol (2.5%). Finally, while none of the subjects experience hot flush after taking 1000 μg of cebranopadol, only a single subject (2.2%) experienced hot flush after cebranopadol 600 μg compared to 11.4% (n=5) after taking oxycodone and 12.8% (n=5) after taking tramadol.

| | | | | | | | Oxycodone | | Tramadol | | | |
|---|---|---|---|---|---|---|---|---|---|---|---|---|
| | | | Cebranopadol | | Cebranopadol | | HCl | | HCl | | | |
| System Organ | Placebo | | 600 μg | | 1000 μg | | 40 mg | | 600 mg | | Total | |
| Class | (N = 45) | | (N = 45) | | (N = 43) | | (N = 44) | | (N = 39) | | (N = 47) | |
| Preferred Term | n (%) | | n (%) | | n (%) | | n (%) | | n (%) | | n (%) | |
| Number of | 7 | (15.6%) | 19 | (42.2%) | 22 | (51.2%) | 25 | (56.8%) | 26 | (66.7%) | 44 | (93.6%) |
| subjects with at | | | | | | | | | | | | |
| least one | | | | | | | | | | | | |
| TEAE | | | | | | | | | | | | |
| Gastrointestinal | 2 | (4.4%) | 9 | (20.0%) | 16 | (37.2%) | 12 | (27.3%) | 14 | (35.9%) | 29 | (61.7%) |
| disorders | | | | | | | | | | | | |
| Nausea | 2 | (4.4%) | 8 | (17.8%) | 15 | (34.9%) | 10 | (22.7%) | 13 | (33.3%) | 28 | (59.6%) |
| Vomiting | 0 | | 7 | (15.6%) | 13 | (30.2%) | 7 | (15.9%) | 10 | (25.6%) | 20 | (42.6%) |
| Hiccups | 0 | | 1 | (2.2%) | 1 | (2.3%) | 3 | (6.8%) | 1 | (2.6%) | 5 | (10.6%) |
| Abdominal pain | 0 | | 1 | (2.2%) | 0 | | 0 | | 0 | | 1 | (2.1%) |
| upper | | | | | | | | | | | | |
| Toothache | 0 | | 0 | | 1 | (2.3%) | 0 | | 0 | | 1 | (2.1%) |
| Uvulitis | 0 | | 0 | | 0 | | 0 | | 1 | (2.6%) | 1 | (2.1%) |
| Nervous system | 5 | (11.1%) | 8 | (17.8%) | 10 | (23.3%) | 10 | (22.7%) | 10 | (25.6%) | 22 | (46.8%) |
| disorders | | | | | | | | | | | | |
| Somnolence | 3 | (6.7%) | 2 | (4.4%) | 6 | (14.0%) | 4 | (9.1%) | 3 | (7.7%) | 10 | (21.3%) |
| Dizziness | 1 | (2.2%) | 3 | (6.7%) | 4 | (9.3%) | 4 | (9.1%) | 1 | (2.6%) | 9 | (19.1%) |
| Headache | 2 | (4.4%) | 3 | (6.7%) | 2 | (4.7%) | 2 | (4.5%) | 3 | (7.7%) | 8 | (17.0%) |

| Generalised tonic- | 0 | 0 | | 0 | | 0 | | 2 | (5.1%) | 2 | (4.3%) |
|---|---|---|---|---|---|---|---|---|---|---|---|
| clonic seizure | | | | | | | | | | | |
| Vertigo | 0 | 0 | | 1 | (2.3%) | 0 | | 1 | (2.6%) | 2 | (4.3%) |
| Tension headache | 0 | 1 | (2.2%) | 0 | | 0 | | 0 | | 1 | (2.1%) |
| Tremor | 0 | 0 | | 1 | (2.3%) | 0 | | 0 | | 1 | (2.1%) |
| Skin and | 0 | 3 | (6.7%) | 3 | (7.0%) | 13 | (29.5%) | 10 | (25.6%) | 20 | (42.6%) |
| subcutaneous | | | | | | | | | | | |
| tissue disorders | | | | | | | | | | | |
| Pruritus | 0 | 2 | (4.4%) | 3 | (7.0%) | 10 | (22.7%) | 6 | (15.4%) | 15 | (31.9%) |

| | | | | | | | | | |
|---|---|---|---|---|---|---|---|---|---|
| Hyperhidrosis | 0 | 0 | 0 | 5 | (11.4%) | 5 | (12.8%) | 9 | (19.1%) |

### Example 2-17: Alternative Cebranopadol Compositions

Cebranopadol was dissolved in solvent at 80° C. The carrier material was added portion wise (0.05 g) at 80° C. until the mixture was a free flowing homogenous powder. The composition was allowed to cool to room temperature (23° C.).

The employed amounts and the resulting solid state of the formulations are specified in Table 1.

| Example | | Solvent | Cebranopadol | | Carrier |
|---|---|---|---|---|---|
| no. | Solvent | [ml] | [mg] | Carrier | [mg] |
| 2 | glycofurol | 0.4 | 10 | Neusilin ® | 200 |
| 3 | glycofurol | 0.4 | 10 | SBA-16 | 650 |
| 4 | glycofurol | 0.4 | 10 | SBA-15 | 200 |
| 5 | glycofurol | 0.4 | 10 | MSU-F | 150 |
| 6 | glycofurol | 0.4 | 10 | MCM-48 | 200 |
| 7 | glycofurol | 0.4 | 10 | A1-MCM- | 200 |
| | | | | 41 | |
| 8 | glycofurol | 0.4 | 10 | Celite | 400 |
| 9 | glycofurol | 0.4 | 10 | Aeroperl ® | 200 |
| | | | | 300 | |
| 10 | diethylene glycol | 0.55 | 10 | Neusilin ® | 250 |
| | monomethyl ether | | | | |
| 11 | diethylene glycol | 0.55 | 10 | SBA-16 | 950 |
| | monomethyl ether | | | | |
| 12 | diethylene glycol | 0.55 | 10 | SBA-15 | 250 |
| | monomethyl ether | | | | |
| 13 | diethylene glycol | 0.55 | 10 | MSU-F | 250 |
| | monomethyl ether | | | | |
| 14 | diethylene glycol | 0.55 | 10 | MCM-48 | 250 |
| | monomethyl ether | | | | |
| 15 | diethylene glycol | 0.55 | 10 | A1-MCM- | 250 |
| | monomethyl ether | | | 41 | |
| 16 | diethylene glycol | 0.55 | 10 | Celite | 400 |
| | monomethyl ether | | | | |
| 17 | diethylene glycol | 0.55 | 10 | Aeroperl ® | 250 |
| | monomethyl ether | | | 300 | |

All patents, patent publications, and other publications listed in this specification are incorporated herein by reference, including US Provisional Patent Application No. 63/387,086, filed Dec. 12, 2022; U.S. Provisional Patent Application No. 63/485,139, filed Feb. 15, 2023 and U.S. Provisional Patent Application No. 63/578,894, filed Aug. 25, 2023. While the invention has been described with reference to a particularly preferred embodiment, it will be appreciated that modifications can be made without departing from the spirit of the invention. Such modifications are intended to fall within the scope of the appended claims.

1. A composition useful in treating a subject, wherein the composition comprises cebranopadol or a pharmaceutically acceptable salt, hydrate, or salt hydrate, which when administered to a subject provides the subject with a therapeutic effect of a pharmaceutically acceptable full mu-agonist and a lower potential for abuse than a pharmaceutically acceptable partial mu-agonist.

2. The composition of claim 1, wherein subject is being treated for pain.

3. The composition of claim 1, wherein the pain is chronic; acute; central; peripheral; neuropathic and/or nociceptive pain.

4. The composition of claim 1, wherein the pain is visceral pain, skeletal pain, and/or nervous pain.

5. The composition of claim 1, wherein the dose is administered daily in an amount of about 10 ug to about 2000 ug cebranopadol.

6. The composition of claim 1, wherein the cebranopadol is a free base.

7. The composition of claim 1, wherein at least 80% of the cebranopadol is in crystal form A.

8. The composition of claim 1, wherein the partial mu-agonist is tramadol.

9.-10. (canceled)

11. A method for treating a subject for pain comprising administering a composition according to claim 1.

12. (canceled)

13. The method of claim 11, wherein the pain is chronic; acute; central; peripheral; neuropathic and/or nociceptive pain.

14. The method of claim 11, wherein the pain is visceral pain, skeletal pain, and/or nervous pain.

15. The method of claim 11, wherein the mu-agonist activity is assessed using a visual analog scale (VAS rating), pupillometry, and/or a Multi-Task Test.

16. The method of claim 11, wherein the dose is administered daily in an amount of about 10 ug to about 2000 ug cebranopadol.

17. The method of claim 11, wherein the cebranopadol is a free base.

18. The method of claim 11, wherein at least 80% of the cebranopadol is in crystal form A.

19. The method any claim 11, wherein the partial mu-agonist is tramadol.

20. A method for treating pain in a patient having nociceptive pain with reduced risk of abuse, said regimen comprising dosing a patient once daily with an immediate release composition comprising cebranopadol or a pharmaceutically acceptable salt thereof.

21.-22. (canceled)

23. The method according to claim 20, wherein at least 80% of the cebranopadol in the composition is in crystal form A.

24. A composition useful for reducing pruritus, hyperhidrosis, feeling hot and/or hot in a subject receiving analgesic treatment for pain, while providing the analgesic therapeutic effect of an opioid, wherein the composition comprises cebranopadol.

25. The composition according to claim 20, wherein the cebranopadol is in free base form.

26. The composition according to claim 21, wherein at least 80% of the cebranopadol in the composition is in crystal form A.

27. A method for reducing pruritus, hyperhidrosis, feeling hot and/or hot in a subject receiving analgesic treatment for pain, while providing the analgesic therapeutic effect of an opioid, comprising administering a composition comprising cebranopadol.

28. The method according to claim 23, wherein cebranopadol is the sole analgesic in the composition.

29. A method for reducing the abuse potential and sides effects of Class II, III and Class IV-opioids and opioid-like analgesics in a patient in need of analgesic treatment, said method comprising dosing a patient once a day with an immediate release composition comprising cebranopadol or a pharmaceutically acceptable salt or hydrate thereof.

30.-38. (canceled)

39. The method according to claim 29, wherein at least 80% of the cebranopadol is crystal form A.

40. A regimen for reducing the abuse potential and sides effects of Class II and Class IV-opioids in a patient susceptible thereto, said method comprising:
(a) discontinuing treatment of a patient with an opioid or opioid agonist which lacks a dual receptor for nociceptin/orphanin FQ peptide (NOP) and μ-opioid peptide (MOP) receptor; and
(b) dosing a patient once a day with an immediate release composition comprising cebranopadol or a pharmaceutically acceptable salt thereof.

41.-48. (canceled)

49. The regimen according to claim 40, wherein at least 80% of the cebranopadol is crystal form A.
